(19) United States
(12) Patent Application Publication
(10) Pub. No.: US 2025/0018088 A1
(43) Pub. Date: Jan. 16, 2025

(54) COMPOSITIONS AND METHODS FOR REPAIRING DAMAGE TO SKELETAL MUSCLE

(71) Applicant: MAYO FOUNDATION FOR MEDICAL EDUCATION AND RESEARCH, Rochester, MN (US)

(72) Inventors: Atta Behfar, Rochester, MN (US); Emanuel C. Trabuco, Rochester, MN (US)

(21) Appl. No.: 18/714,438

(22) PCT Filed: Dec. 1, 2022
(86) PCT No.: PCT/US2022/051527

§ 371 Date: May 29, 2024

Related U.S. Application Data
(60) Provisional application No. 63/284,989, filed on Dec. 1, 2021.

(51) Int. Cl. A61L 27/54; A61K 35/19; A61L 27/24; A61L 27/52; A61P 21/00

(52) U.S. Cl. CPC A61L 27/54; A61K 35/19; A61L 27/24; A61L 27/52; A61P 21/00; A61L 2430/30

(57) ABSTRACT

A method of treating injured skeletal muscle tissue generally includes applying to injured skeletal muscle tissue a therapeutic composition that includes a purified exosome product (PEP) and a pharmaceutically acceptable carrier. In one or more embodiments, the method includes applying from 1×1011 PEP exosomes to 1×1013 PEP exosomes to the injured skeletal muscle. In one or more embodiments, the therapeutic composition further includes a supportive matrix such as a collagen scaffold, a tissue sealant, fibrin glue, or a hydrogel.

### CROSS-REFERENCE TO RELATED APPLICATIONS

This application claims the benefit of U.S. Provisional Patent Application Ser. No. 63/284,989, filed on Dec. 1, 2021, which is incorporated by reference herein in its entirety.

### SUMMARY

This disclosure describes, in one aspect, a method of treating injured skeletal muscle tissue. Generally, the method includes applying to injured skeletal muscle tissue a therapeutic composition that includes a purified exosome product (PEP) and a pharmaceutically acceptable carrier.

In one or more embodiments, the PEP includes spherical or spheroid exosomes having a diameter no greater than 300 nm.

In one or more embodiments, the PEP includes from 1% to 20% CD63⁻ exosomes and from 80% to 99% CD63⁺ exosomes. In other embodiments, the PEP includes at least 50% CD63⁻ exosomes.

In one or more embodiments, the method includes applying from 1×10¹¹ PEP exosomes to 1×10¹³ PEP exosomes to the injured skeletal muscle.

In one or more embodiments, the therapeutic composition further includes a supportive matrix such as a collagen scaffold, a tissue sealant, fibrin glue, or a hydrogel.

In one or more embodiments, the therapeutic composition is applied in an amount effective to increase expression of NF-κB in the injured skeletal muscle tissue compared to injured skeletal muscle tissue treated without the therapeutic composition.

In one or more embodiments, the therapeutic composition is applied in an amount effective to increase expression of PD-L1 in the injured skeletal muscle tissue compared to injured skeletal muscle tissue treated without the therapeutic composition.

In one or more embodiments, the therapeutic composition is applied in an amount effective to increase polarization toward M2 macrophages in the injured skeletal muscle tissue compared to injured skeletal muscle tissue treated without the therapeutic composition.

In one or more embodiments, the injured skeletal muscle tissue comprises muscle of the urethral sphincter.

In one or more embodiments, at least a portion of the PEP exosomes include PD-L1, NF-κB, or both PD-L1 and NF-κB.

In one or more embodiments, at least a portion of the PEP exosomes include CD63, CD9, flotillin, or any combination of two or more of the foregoing.

In one or more embodiments, the therapeutic composition is applied in an amount effective to increase cellular proliferation in the injured skeletal muscle tissue compared to injured skeletal muscle tissue treated without the therapeutic composition.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Purified Exosome Product (PEP) Characterization, Quantification and in vitro Human Skeletal Muscle Myoblasts (HSMM) Culturing with Increasing Concentration of PEP. (A) NanoSight nanoparticle analysis of size distribution and concentration of PEP diluted in phosphate buffered saline 1:1000. (B) Transmission electron microscopy of PEP. Scale=200 nm. (C) Western blot probing for CD63, CD9, and Flotillin-1 in three PEP preparations. (D) Western blot comparison of NF-κB p65 and PD-L1 levels in 3 PEP preparations versus adipose-derived mesenchymal stem cell conditioned media (AMSC-CM). IncuCyte proliferation (E), chemotaxis (F) and wound-scratch (G) analyses of HSMM grown with increasing concentration of PEP ranging from 1.25×10¹¹ exosomes/mL to 5×10¹¹ exosomes/mL versus serum free media and media supplemented with 10% fetal bovine serum (FBS).

FIG. 2. Purified Exosome Product (PEP) Characterization, Quantification and in vitro Human Skeletal Muscle Myoblasts (HSMM) Culturing with Increasing Concentration of PEP. Nuclei counterstained with DAPI (blue). (A) Representative immunocytochemistry of a 96-hour time-course of HSMM cultured with serum-free media (Control) or serum free media plus 2.5×10¹¹ exosomes/mL PEP (PEP). Immunostaining for MyoD (B) ImageJ blind quantification of immunostaining for MyoD+ Area/DAPI+ Area, scale=100 μm. (C) Representative immunocytochemistry of a 96 hour time-course of HSMM cultured with serum-free media (Control) or serum free media plus 2.5×10¹¹ exosomes/mL PEP (PEP). (D) ImageJ blind quantification of immunostaining for Pax7+ Area/DAPI+ Area. N.S.=Not significant. * p<0.05. ** p<0.005.

FIG. 3. Purified Exosome Product (PEP) Characterization, Quantification and in vitro Human Skeletal Muscle Myoblasts (HSMM) Culturing with Increasing Concentration of PEP. Nuclei counterstained with DAPI (blue). (A) Representative immunocytochemistry of a 96-hour time-course of HSMM cultured with serum free media (Control) or serum free media plus 2.5×10¹¹ exosomes/mL PEP (PEP). Immunostaining for MyoD Myosin Heavy Chain (MHC), scale=20 μm. (B) ImageJ blind quantification of Myosin Heavy Chain+ Area/DAPI object count, relative to day zero. N.S.=Not significant. * p<0.05. ** p<0.005.

FIG. 4. Resveratrol dose-dependent inhibition of PEP-mediated HSMM proliferation. Representative live cell images of HSMM cultured with basal media, 2.5×10¹¹ exosomes/mL PEP, and 2.5×10¹¹ exosomes/mL PEP with increasing concentrations of resveratrol. Scale=400 μm. (N.S.=Not significant, * p<0.05, ** p<0.0001).

FIG. 5. Resveratrol dose-dependent inhibition of PEP-mediated HSMM proliferation. (A) Representative proliferation growth curves of HSMM cultured with basal media, 2.5×10¹¹ exosomes/mL PEP, and 2.5×10¹¹ exosomes/mL PEP with increasing concentrations of resveratrol. Scale=400 μm. (N.S.=Not significant, * p<0.05, ** p<0.0001). (B) Resveratrol dose-dependent inhibition of HSMM proliferation. Data points from corresponding live cell analysis of respective growth conditions (FIG. 4 and FIG. 5A) at t=72 hours.

FIG. 6. Resveratrol dose-dependent inhibition of PEP-mediated HSMM proliferation. (A) Western blot of NF-κB p65 expression in HSMM cells cultured with media supplemented with 2.5×10¹¹ exosomes/mL PEP alone or with increasing concentrations of resveratrol. (B) Basal NF-κB p65 protein expression compared to the expression obtained from cells cultured in 2.5×10¹¹ exosomes/mL PEP alone or with increasing resveratrol concentrations (all comparisons to PEP, * p<0.05, ** p<0.0001).

FIG. 7. Schematic of hypothesized mechanism of PEP donation and role of NF-κB p65 on skeletal muscle proliferation.

FIG. 8. Repair of rat volumetric muscle loss latissimus dorsi defect with PEP. (A) Study timeline. (B) Schematic of latissimus dorsi volumetric muscle loss rat model. (C) Scanning electron microscopy of TISSEEL (Baxter International, Inc., Deerfield, IL; Left) and TISSEEL reconstituted with 1×10¹² exosomes/mL PEP (Right). Scale=500 nm.

FIG. 9. Repair of rat volumetric muscle loss latissimus dorsi defect with PEP. TISSEEL release assay to show sustained exosome release. Triplicates are reported every 24 hours and serum free media was replaced daily.

FIG. 10. Repair of rat volumetric muscle loss latissimus dorsi defect with PEP. (A) Representative photographs of gross healing at injury site and (B) Masson's Trichrome staining of VML sites treated with saline, TISSEEL, and TISSEEL reconstituted with 1×10¹² exosomes/mL PEP. Dashed region highlights area of biopsy punch injury. Images obtained with 10× magnification.

FIG. 11. Representative sections of immunostaining of VML sites treated with saline sham, TISSEEL, or TISSEEL reconstituted with 1×10¹² exosomes/mL PEP. Nuclei were counterstained with DAPI (blue), skeletal muscle protein desmin (red), and cell proliferation with EdU (cyan). Scale Bar=500 μm.

FIG. 12. ImageJ blind quantification of de novo skeletal muscle proliferation by calculating the proportion of EdU positive cells per mm² of desmin positive area (red). * p<0.05. ** p<0.005.

FIG. 13. Creation of porcine SUI model and urethral pressures following repair of urethral sphincter lesion with PEP. (A) Study timeline. (B) Schematic of cystoscopic induced transurethral full thickness, focal urethral sphincter defect to create the porcine SUI model. Note the apposition of the urethral sphincter muscle to the anterior surface of the vagina and urethra. (C) Schematic of gross appearance of the urethral sphincter defect at day 0, prior to injecting the intervention (Day 7), and prior to euthanasia (Day 42). (D) Modified Medspira mCompass pressure catheter highlighting stabilization of the catheter at the bladder neck (grey) and movable pressure sensor (green) used to obtain pressures along the length of the pig's vagina and urethra.

FIG. 14. Creation of porcine SUI model and urethral pressures following repair of urethral sphincter lesion with PEP. (A) Scanning electron microscopy of collagen and collagen reconstituted with 1×10¹² exosomes/mL PEP. Scale=1 μm. (B) Collagen release assay to show sustained exosome release (n=3).

FIG. 15. Creation of porcine SUI model and urethral pressures following repair of urethral sphincter lesion with PEP. Representative cystoscopic images of the urethral sphincter lesion on day 7 prior to delivering the intervention and on day 42 prior to euthanasia. Arrows point to the site of the urethral sphincter defect.

FIG. 16. Creation of porcine SUI model and urethral pressures following repair of urethral sphincter lesion with PEP. Graph of mean urethral pressures pre- and post-injury on day 0 (n=10), pre-injection on day 7 (n=10) and prior to euthanasia on day 42. Collagen=injection of collagen; PEP=injection of collagen reconstituted with 1×10¹² exosomes/mL PEP. D42 Control=urethral sphincter defect repaired with collagen (n=4); Day 42 PEP=urethral sphincter defect repaired with collagen reconstituted with 1×10¹² exosomes/mL PEP (n=6). * p<5×10⁻⁸; ** p<5×10⁻¹⁴; #p<1×10⁻⁵; ##p<1×10⁻⁷; p<5×10⁻¹⁰; #p<1×10⁻¹³.

FIG. 17. Immunohistochemical analysis of PEP-induced repair of urethral sphincter injury. Representative sections of urethral sphincter lesion repaired with collagen or collagen reconstituted with 1×10¹² exosomes/mL PEP and intact urethra stained. (A) Nuclei were counterstained with DAPI (blue), skeletal muscle with desmin (red), and EdU (white). Scale Bar=100 μm. Magnified view of representative sections of intact urethra (Intact) and urethral sphincter lesion repaired with collagen (Collagen) or collagen reconstituted with 1×10¹² exosomes/mL PEP (PEP). (n=9, 18, 15 respectively).

FIG. 18. Immunohistochemical analysis of PEP-induced repair of urethral sphincter injury. Representative sections of urethral sphincter lesion repaired with collagen or collagen reconstituted with 1×10¹² exosomes/mL PEP and intact urethra stained. Tissue was stained for PD-L1 (green), p65 NF-κB (red), and DAPI (blue). Merge overlay highlights PEP treated samples contained co-localization of PD-L1/NF-κB p65 staining of tissues. (n=15, 15, 15), scale=20 μm.

FIG. 19. Immunohistochemical analysis of PEP-induced repair of urethral sphincter injury. Representative sections of urethral sphincter lesion repaired with collagen or collagen reconstituted with 1×10¹² exosomes/mL PEP and intact urethra stained. (A) ImageJ blinded quantification of de novo skeletal muscle proliferation estimated by calculating the number of EdU positive cells per mm² of desmin positive area. (B) ImageJ blinded quantification of PD-L1 expression by analysis of PD-L1 positive tissue area per total tissue area. (C) ImageJ blinded quantification of NF-κB p65 expression by analysis of NF-κB p65+ tissue area per total tissue area. * p<0.05.

FIG. 20. PEP Induces M2 macrophage polarization in a porcine SUI model. Representative sections of urethral sphincter lesion repaired with collagen, collagen reconstituted with 1×10¹² exosomes/mL PEP or intact urethra. Tissues were stained for general macrophage antigen (Mϕ; green), CD163 M2 specific macrophages (red), and DAPI (blue). Merge overlay highlights PEP samples contained co-localization of Mϕ+/CD163+ staining of tissues. Representative sections of intact urethra (Intact) and urethral sphincter lesion repaired with collagen (Collagen) or collagen reconstituted with 1×10¹² exosomes/mL PEP (PEP). (n=12, 15, 20 respectively), scale=20 μm.

FIG. 21. PEP Induces M2 macrophage polarization in a porcine SUI model. Representative sections of urethral sphincter lesion repaired with collagen, collagen reconstituted with 1×10¹² exosomes/mL PEP or intact urethra. ImageJ blinded quantification of Mϕ and M2 staining. M2:M1 ratio was determined from the comparison of image area of M2:(Mϕ−M2). A Haldane correction was utilized to account for division of 0 in M2:M1 ratio. The correction adds 0.5 to all values in the list to allow for the calculation to not yield an error. The graph represents the log base 2 ratio of M2:M1 macrophages. Dotted line at y=0 shows the value at which the ratio would be equal. The log base 2 scale shows a M2:M1>1 at values Log 2>0. * p<0.05.

FIG. 22. Characterization of cGMP PEP preparations. (A) Atomic force microscope comparing platelet-conditioned medium EV isolation using centrifugation versus the PEP process, scalebar embedded in the image. (B) Representative image from Single-particle interferometric reflectance imaging sensing (SP-IRIS) analysis for presence of surface CD41a, CD9, CD63 and CD81 tetraspanins. (C) Graphical representation of the SP-IRIS analysis. (D) Quantitation of CD9, CD63 and CD81 on a CD41a captured plate documented vast majority of PEP as CD41a/CD9 positive, with smaller representation from CD63 and background CD81. Data presented as mean±stdev. N=3 separate CGMP manufactured PEP lots. (E) Pie chart representation of the exosome tetraspanin surface marker profile of CD41a captured PEP exosomes.

### DETAILED DESCRIPTION OF ILLUSTRATIVE EMBODIMENTS

This disclosure describes compositions and methods for repairing injured or damaged skeletal muscle tissue. Generally, the compositions include a purified exosome product (PEP). When applied to damaged or injured skeletal muscle tissue, the PEP composition promotes healing and/or repair of the injured or damaged skeletal muscle tissue.

While described below in the context of exemplary embodiments in which the damaged skeletal muscle is muscle of the urethral sphincter or latissimus dorsi, the methods described herein may involve the repair of damage to any skeletal muscle. Other exemplary skeletal muscles that may be repaired using the methods described herein include, but are not limited to, the deltoid, trapezius, sternocleidomastoid, abdominals, obliques, and adductors.

Urinary incontinence afflicts up to 40% of adult women in the United States. Stress urinary incontinence (SUI), accounts for approximately one-third of these cases, precipitating ˜200,000 surgical procedures annually. Continence is maintained through the interplay of suburethral support and urethral sphincter coaptation, particularly during activities which increase intra-abdominal pressure. Currently, surgical correction of SUI focuses on reestablishment of suburethral support. However, mesh-based repairs may be associated with foreign body reaction and poor localized tissue healing, which leads to mesh exposure, prompting pursuit of technologies that restore external urethral sphincter function and limit surgical risk.

This disclosure describes the use of a purified exosome product (PEP) engineered to be enriched for NF-κB-positive and PD-L1-positive exosomes as a clinical-grade off-the-shelf biologic to drive skeletal muscle regeneration. NF-κB and PD-L1 are proteins that play a role in suppressing the immune system. The PD-L1⁺/NF-κB⁺ population of PEP exosomes may promote tissue repair more than PD-L1⁻/NF-κB⁻ exosomes. In one or more embodiments, the PEP exosomes described herein are positive for CD63, CD9, and/or flotillin.

Application of PEP to skeletal muscle satellites in vitro, as described herein, drove proliferation and differentiation in an NF-κB-dependent fashion. In vivo, suspension of PEP exosomes in a hydrogel achieved sustained exosome release, inducing functional restoration of the external urethral sphincter associated with new skeletal muscle formation, and polarization of local macrophages towards the regenerative M2 phenotype. The data presented herein support the use of PEP as an off-the-shelf, cell independent exosome-based approach to restore skeletal muscle function and provide non-surgical management of SUI.

In one or more embodiments, a composition including PEP forms a hydrogel. The composition may form a hydrogel before application to an area to be treated, or the composition may form a hydrogel after application to an area to be treated. In one or more certain embodiments, the composition may form a hydrogel upon incubation at an incubation temperature. The incubation temperature may be, for example, at least 30° C., at least 31° C., at least 32° C., at least 33° C., at least 34° C., at least 35° C., at least 36° C., or at least 37° C. The incubation temperature may be, for example, at most 45° C., at most 44° C., at most 43° C., at most 42° C., at most 41° C., at most 40° C., at most 39° C., or at most 38° C. The incubation temperature may be 33° C. to 40° C., such as 35° C. to 39° C., or 37° C.

It may be desirable for the composition to form a hydrogel after incubation on a desired treatment site for a given amount of time. If the composition forms a hydrogel too rapidly, it may be more challenging to deliver or distribute the composition within a treatment site. In one or more embodiments, the composition may form a hydrogel, for example, one minute, two minutes, three minutes, four minutes, five minutes, eight minutes, 10 minutes, 12 minutes, or 15 minutes after application.

This disclosure describes the use of a purified exosome product (PEP) for treating skeletal muscle such as, for example, treating skeletal muscle of the urethral sphincter to treat stress urinary incontinence. The interplay of suburethral support and urethral sphincter contraction is typically required to achieve optimal coaptation and maintain continence during activities that result in increased intra-abdominal pressure. Events such as vaginal delivery can disrupt these coordinated events and compromise continence. Most surgical approaches to correct SUI have focused on re-establishing suburethral support with minimal attention paid to restoring urethral sphincter function. For example, the midurethral sling surgery is a minimally invasive procedure that uses a narrow strip of polypropylene mesh placed suburethrally to support the urethra, but does not address sphincter function. Concerns over mesh-based treatment options have emphasized a need for safer and effective non-surgical and non-mesh-based treatment options. As noted above, while described below in the context of an exemplary embodiment in which the damaged skeletal muscle is muscle of the urethral sphincter, the compositions and methods described herein may involve the repair of damage to any skeletal muscle.

The PEP was prepared from a platelet population maintained within a suspension culture to produce a CD63⁺/CD9⁺/Flotillin⁺ exosome population enriched for NF-κB and PD-L1. Treating muscle myoblasts in vitro with PEP exosomes induced chemotaxis, proliferation, and differentiation in a NF-κB-dependent fashion. In vivo, treatment with PEP exosomes induced repair of skeletal muscle in a rat model of volumetric muscle loss. In a porcine SUI model, minimally invasive delivery of PEP resulted in functional restoration of the external sphincter associated with augmented muscle proliferation, local upregulation of NF-κB and PD-L1, and polarization of local macrophages towards the M2 fate. The data presented herein provide mechanistic and translational support for the use of an acellular biologic able to induce local progenitor migration and differentiation following minimally invasive delivery into a porcine model of SUI.

Production of purified exosome product (PEP) involves separating plasma from blood, isolating a solution of exosomes from separated plasma with filtration and centrifugation. PEP is fully characterized and methods for preparing PEP are described in International Patent Application No. PCT/US2018/065627 (published as International Publication No. WO 2019/118817), U.S. Patent Publication No. 2021/0169812 A1, and U.S. Pat. No. 10,596,123, each of which is incorporated by reference herein in its entirety. Typically, the blood is from a human source, however, other blood sources are certainly contemplated.

Briefly, PEP is a purified exosome product prepared using a cryodesiccation step that produces a product having a structure that is distinct from exosomes prepared using conventional methods. For example, PEP typically has a spherical or spheroidal structure and an intact lipid bilayer rather than a crystalline structure that results from the reaggregation of lipids of the exosome lipid bilayer after exosomes are disrupted during convention exosome preparation methods. The spherical or spheroid exosome structures generally have a diameter of no more than 300 nm. Typically, a PEP preparation contains spherical or spheroid exosome structures that have a relatively narrow size distribution. In some preparations, PEP includes spherical or spheroidal exosome structures with a mean diameter of about 110 nm±90 nm, with most of the exosome structures having a mean diameter of 110 nm±50 nm such as, for example, 110 nm±30 nm.

An unmodified PEP preparation—i.e., a PEP preparation whose character is unchanged by sorting or segregating populations of exosomes in the preparation-naturally includes a mixture of CD63⁺ and CD63⁻ exosomes. Because CD63⁻ exosomes can inhibit unrestrained cell growth, an unmodified PEP preparation that naturally includes CD63⁺ and CD63⁻ exosomes can both stimulate cell growth for wound repair and/or tissue regeneration and limit unrestrained cell growth.

Further, by sorting CD63⁺ exosomes, one can control the ratio of CD63⁺ exosomes to CD63⁻ exosomes in a PEP product by removing CD63⁺ exosomes from the naturally-isolated PEP preparation, then adding back a desired amount of CD63⁺ exosomes. In one or more embodiments, a PEP preparation can have only CD63⁻ exosomes.

In one or more embodiments, a PEP preparation can have both CD63⁺ exosomes and CD63⁻ exosomes. The ratio of CD63⁺ exosomes to CD63⁻ exosomes can vary depending, at least in part, on the quantity of cell growth desired in a particular application. For example, a CD63⁺/CD63⁻ exosome ratio provides desired cell growth induced by the CD63⁺ exosomes and inhibition of cell growth provided by the CD63⁻ exosomes achieved via cell-contact inhibition. In certain scenarios, such as in tissues where non-adherent cells exist (e.g., blood derived components), this ratio may be adjusted to provide an appropriate balance of cell growth or cell inhibition for the tissue being treated. Since cell-to-cell contact is not a cue in, for example, tissue with non-adherent cells, one may reduce the CD63⁺ exosome ratio to avoid uncontrolled cell growth. Conversely, if there is a desire to expand out a clonal population of cells, such as in allogeneic cell-based therapy or immunotherapy, one can increase the ratio of CD63⁺ exosomes to ensure that a large population of cells can be derived from a very small source.

Thus, in one or more embodiments, the ratio of CD63⁺ exosomes to CD63⁻ exosomes in a PEP preparation may be at least 1:1, at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, or at least 16:1. In one or more embodiments, the ratio of CD63⁺ exosomes to CD63⁻ exosomes in a PEP preparation may be at most 15:1, at most 16:1, at most 17:1, at most 18:1, at most 19:1, at most 20:1, at most 25:1, or at most 30:1. For example, the ratio of CD63⁺ exosomes to CD63⁻ exosomes may be between 1:1 to 30:1, 2:1 to 20:1, 4:1 to 15:1, or 8:1 to 10:1. In one or more certain embodiments, native PEP, e.g., PEP with an unmodified ratio of CD63⁺ exosomes to CD63⁻ exosomes may be used.

### Exosome Characterization

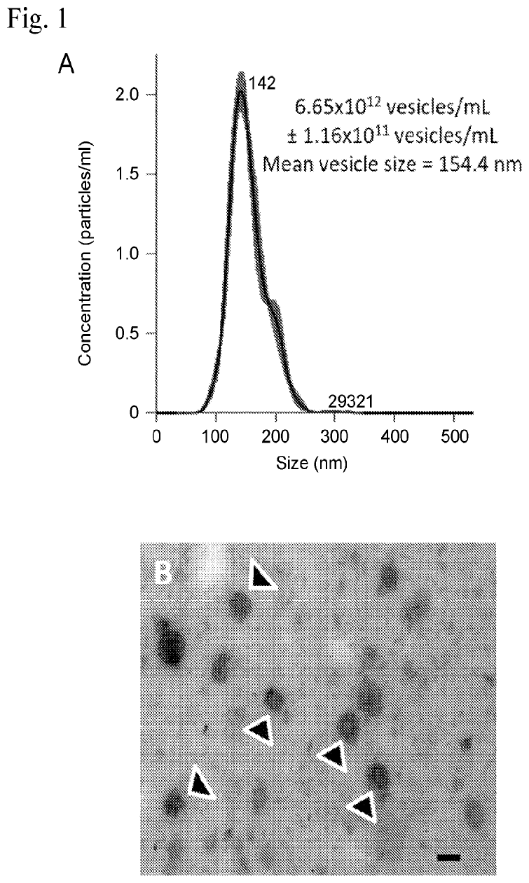

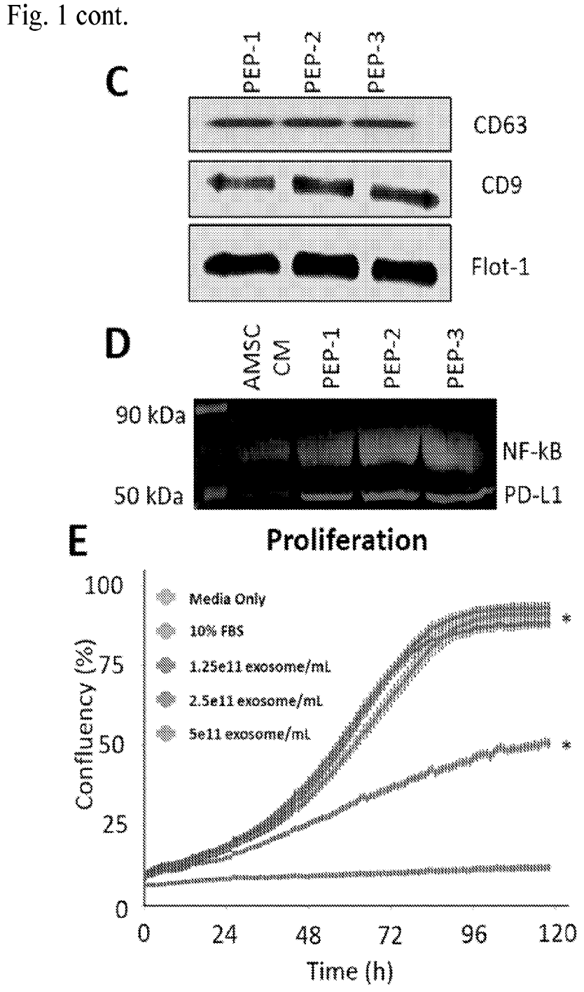

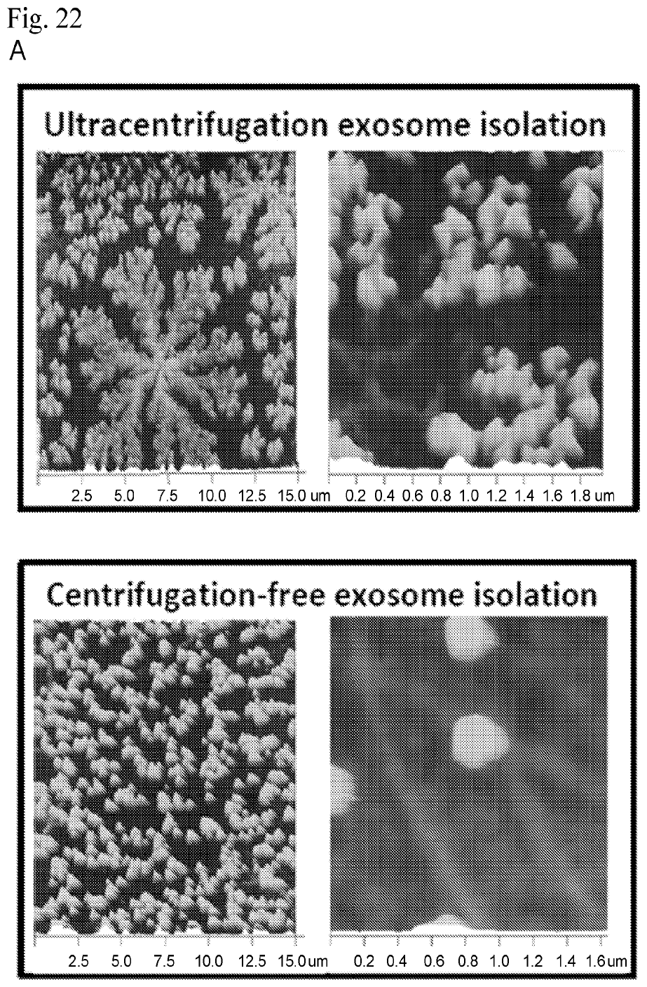

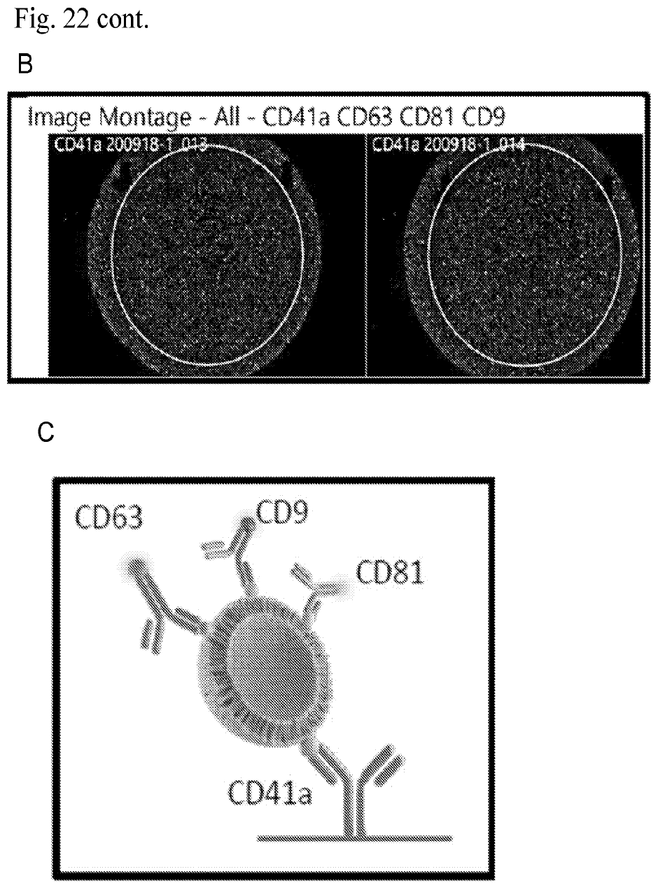

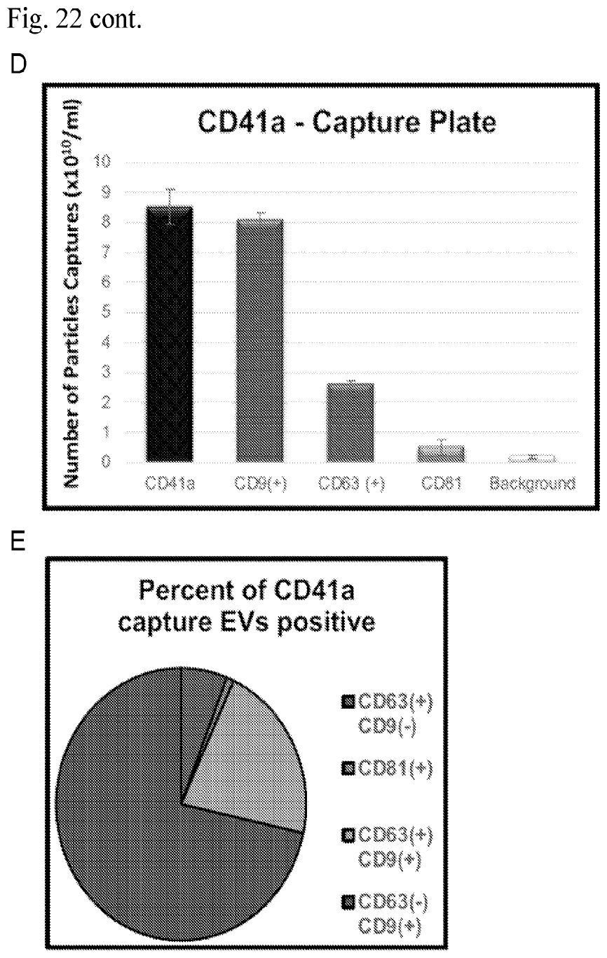

Extracellular vesicles engineered from platelet suspension cultures were assessed for size, lipid bilayer, and markers conformant with exosome populations. (FIG. 1A-C). NanoSight characterization of distinct platelet exosome preparations (PEP) revealed particle concentration of approximately 6.65×10¹² particles/mL and a size mean of ˜154.4 nm, consistent with exosomal populations (FIG. 1A). Electron microscopy of PEP confirmed the NanoSight vesicle size distribution, with the majority of the purified vesicles being approximately 100-150 nm in diameter (FIG. 1i). Western blot analysis showed consistent expression of CD63, CD9, and Flotillin-1, each of which is an established exosomal marker, in three separate clinical preparations of PEP (FIG. 1C). PEP preparations were documented to be enriched for NF-κB p65 (2.41-fold; p<0.05) and PD-L1 (4.98-fold; p<0.005) compared to the levels seen in exosomes purified from adipose-derived mesenchymal stem cells conditioned media (AMSC CM; FIG. 1D). Atomic-force microscopy of EVs purified from platelets using ultracentrifugation document disruption of the phospholipid bilayer, which was preserved with avoidance of high sheer stress purification (FIG. 22A). To confirm derived EVs are platelet exosome preparations (PEP), there were evaluated for presence of concomitant Integrin α2b (CD41) and CD9 expression using single-particle interferometric reflectance imaging sensing (SP-IRIS) analysis of relative quantity of CD63 (6.67±0.58%), CD9 (76.0±0.0%), CD81 (1.0±0.0%), and CD9/CD63 (16.67±0.58%) within CD41α positive exosomes (FIG. 22).

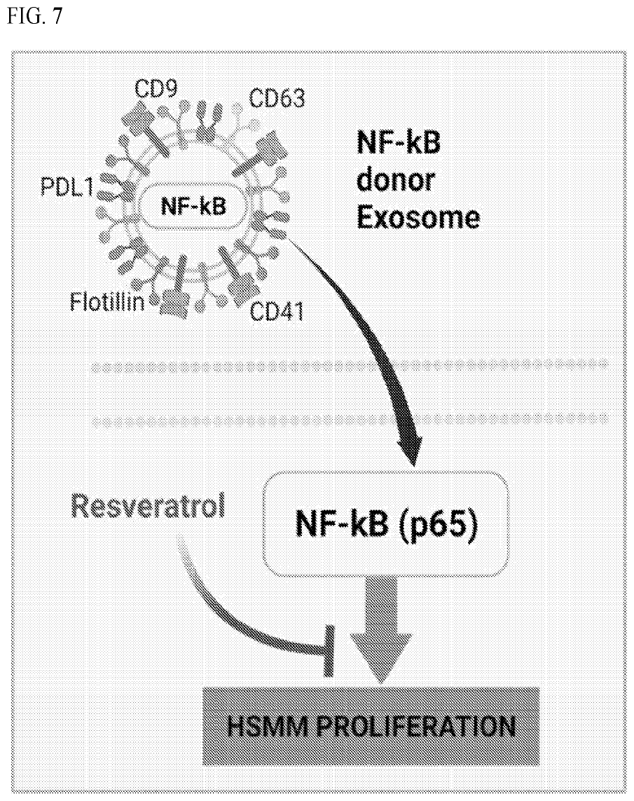

In one or more embodiments, PEP exosomes used in the methods and compositions described herein may be positive for one or more protein markers. Presence of protein markers may be measured by, for example, Western blotting, enzyme-linked immunosorbent assay (ELISA), flow cytometry, dot blotting, or any other suitable molecular biology assay. In one or more embodiments, the PEP exosomes may be positive for PD-L1, CD9, flotillin, NF-κB, or a combination thereof. In one or more embodiments described herein, a PEP preparation may be enriched for exosomes including known anti-inflammatory proteins. Proteins included in a PEP preparation may be on the lipid membrane, or may be contained within the exosome itself. A schematic of a PEP exosome including several proteins on the surface and one within the exosome is shown in FIG. 7.

Presence of certain proteins in a PEP preparation may be the result of, for example, expression of the protein by the donor from which the PEP was derived. Additionally or alternatively, recombinantly expressed proteins may be added to a PEP preparation.

Human Skeletal Muscle Myoblast (HSMM) Co-Cultured with PEP Shows Dose Dependent Growth, Chemotaxis, Cell Migration, and Skeletal Muscle Differentiation

Myoblasts grown with PEP achieved higher percent confluency compared to serum free media and media supplemented with 10% fetal bovine serum (FBS) (FIG. 1E). At PEP concentrations higher than 1.25×10¹¹ exosomes/mL, myoblasts grew to over 90% confluency compared to only ˜50% confluency achieved with standard growth media with 10% FBS. Similarly, a dual chamber migration assay showed that PEP concentrations greater than 2.5×10¹¹ exosomes/mL significantly decreased the total phase area in the top chamber, suggesting that PEP affects myoblast chemotaxis (FIG. 1F). This was validated in a scratch assay showing enhanced wound confluency in PEP treated (≥1.25×10¹¹ exosomes/ml) conditions versus FBS (FIG. 1G).

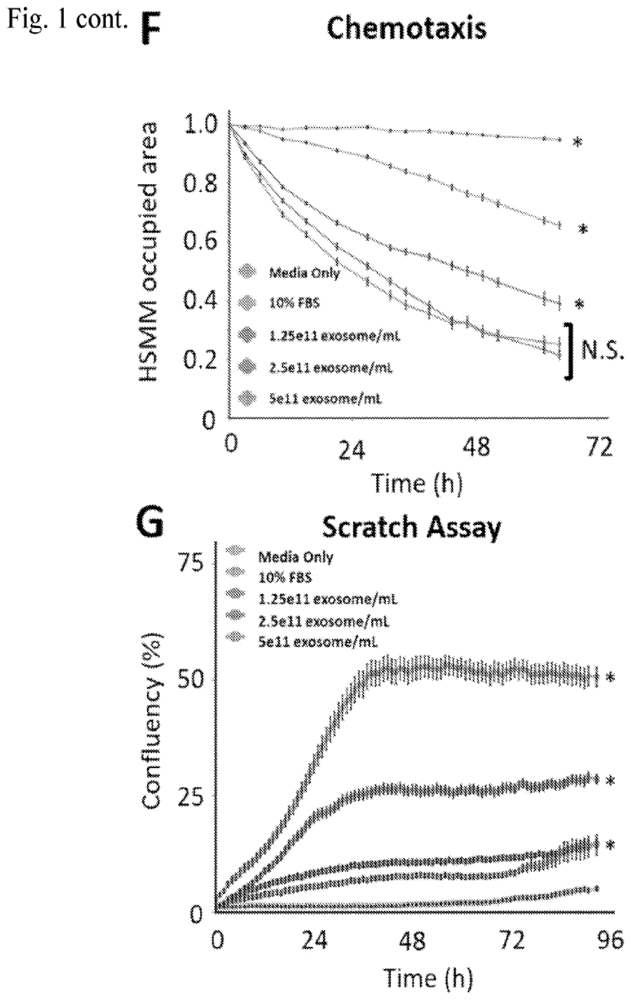

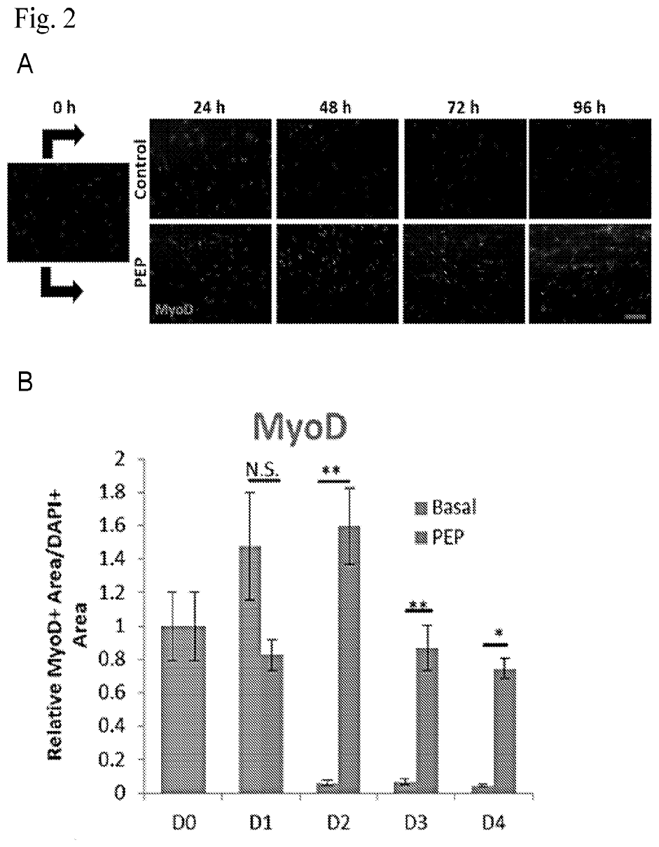

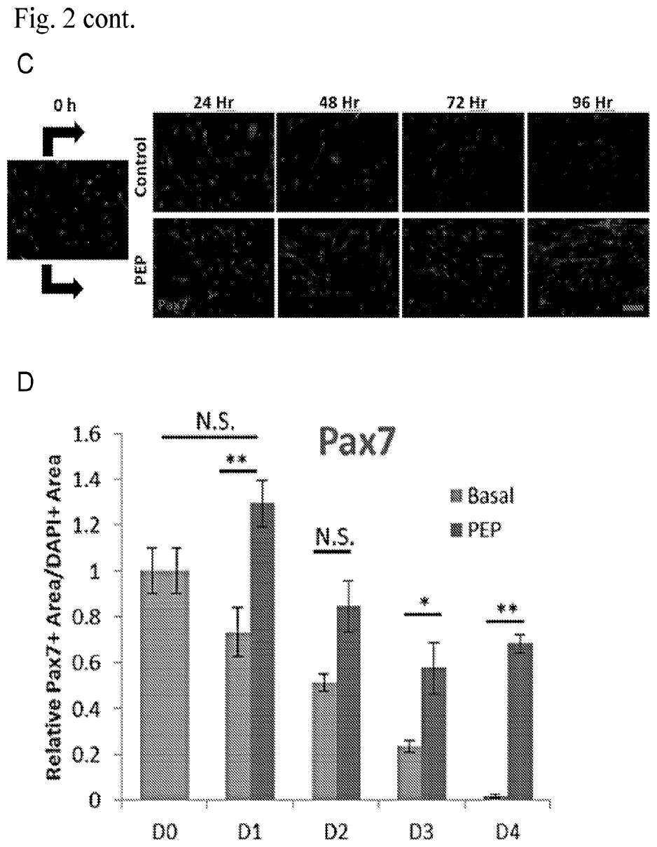

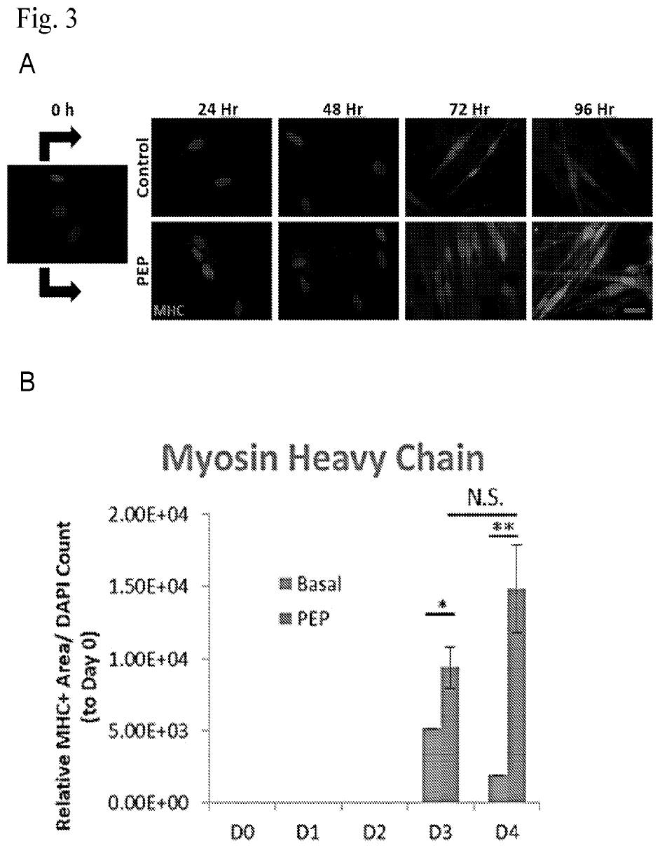

Myogenic lineage maturation from satellite cells to multinucleated myotubes occurred after treatment with 2.5×10¹¹ exosomes/ml of PEP (FIG. 2, FIG. 3). Specifically, myogenic lineage from myoblasts (identified by expression of Pax7 and MyoD) to myotubes (identified by expression of myosin heavy chain (MHC, Myosin Heavy Chain+) was identified after 96 hours by culturing HSMM with PEP (FIG. 2, FIG. 3). By day 4, in contrast to basal media (1949±1106, pixel intensity/area), PEP was able to induce significant MHC induction (14,859±3049, pixel intensity/area, p<0.01), to drive myogenic lineage maturation (FIG. 3A,B).

### Resveratrol Inhibits PEP-Mediated Human Skeletal Muscle Myoblast (HSMM) Proliferation

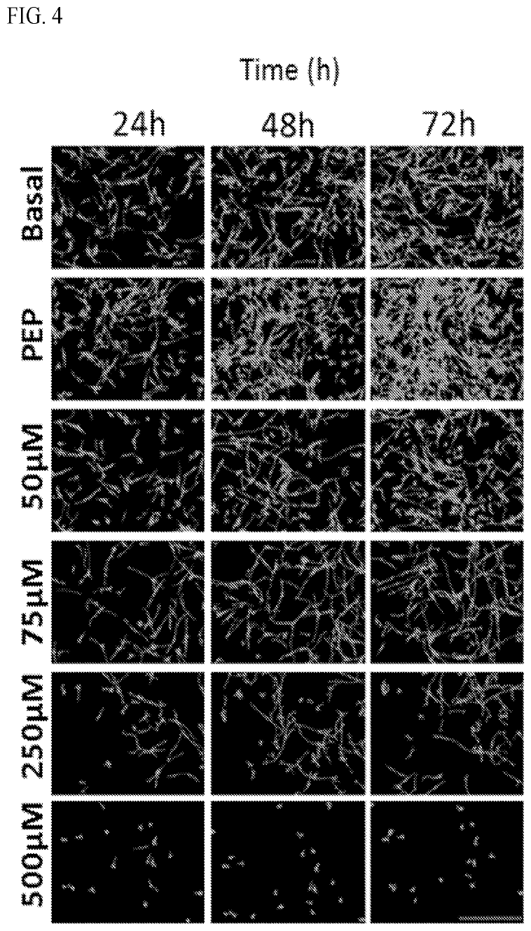

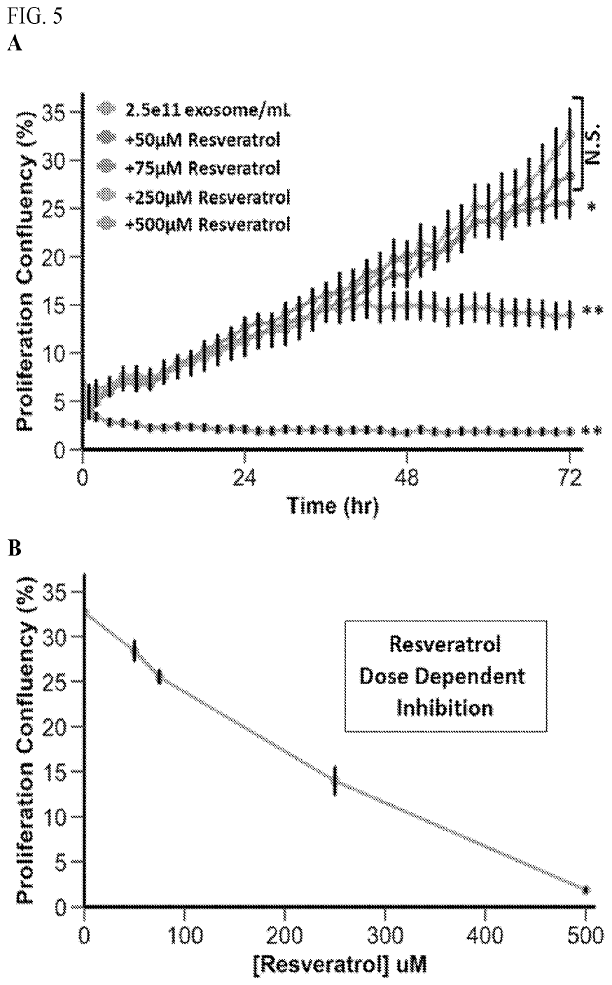

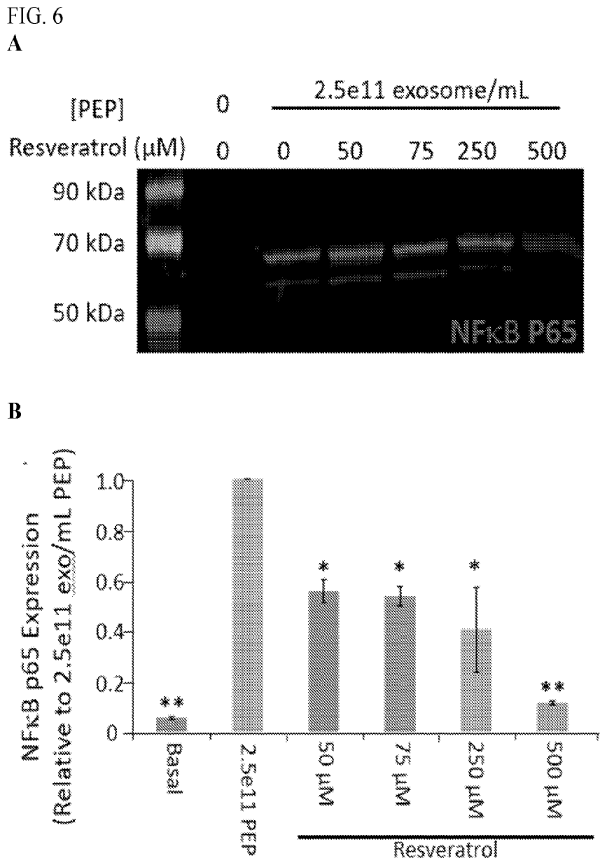

To assess whether PEP donation of NF-κB p65 was responsible for mediating myogenic proliferation and differentiation, HSMM proliferation, cell count, and morphology analysis with PEP alone or in combination with increasing concentrations of resveratrol, a known inhibitor of NF-κB p65, were performed. Myoblast proliferation was inhibited by resveratrol in a dose-dependent fashion. 250 μM resveratrol inhibited proliferation by approximately 50%, and 500 μM resveratrol inhibited proliferation by almost 100% (PEP vs. 250 μM=p<0.0001, PEP vs. 500 μM=p<0.0001; FIG. 4, FIG. 5A). While myoblast growth was not significantly inhibited when treated with only PEP and with low doses of resveratrol (e.g., 50 μM), growth rates declined at higher resveratrol concentrations (FIG. 5B). Western blot analysis of cells grown in each condition showed increased NF-κB p65 (a subunit of NF-κB) levels in PEP-treated HSMM with a dose dependent decrease in NF-κB expression following resveratrol treatment (FIG. 6A,B). These findings confirm that PEP increases expression of NF-κB in HSMM NF-κB p65 levels and the importance of this mitogenic factor in driving skeletal myoblast growth (FIG. 7).

Repair of Rat Volumetric Muscle Loss (VML) Latissimus Dorsi Defect with Purified Exosome Product Enhances Skeletal Muscle Regeneration

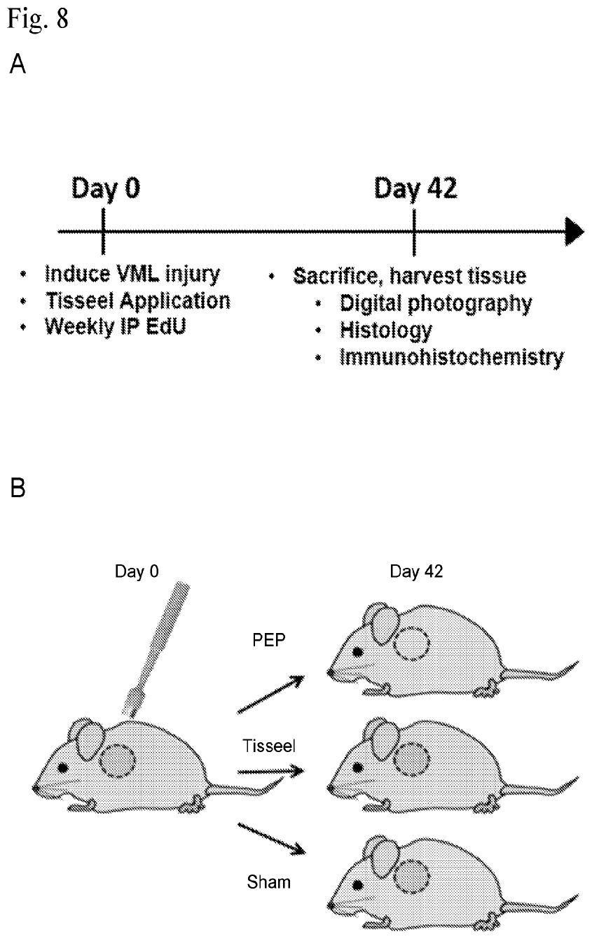

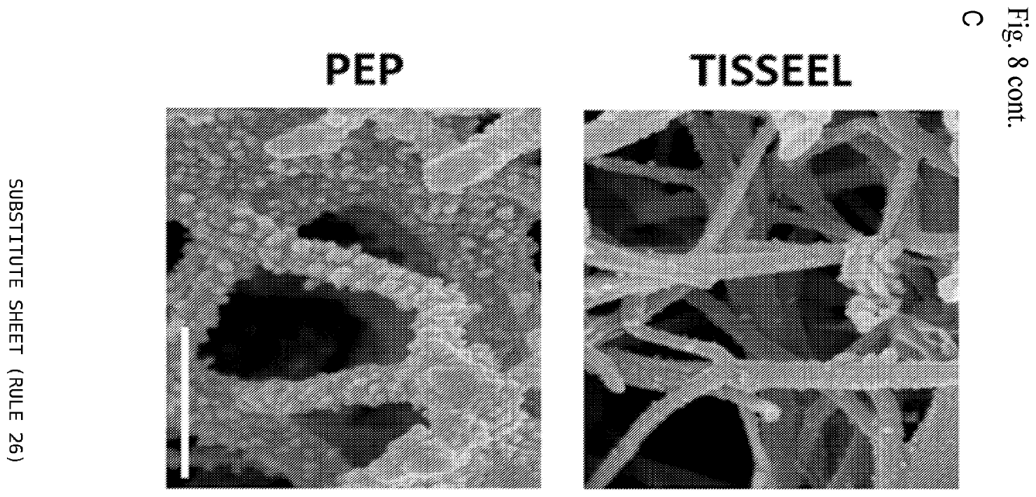

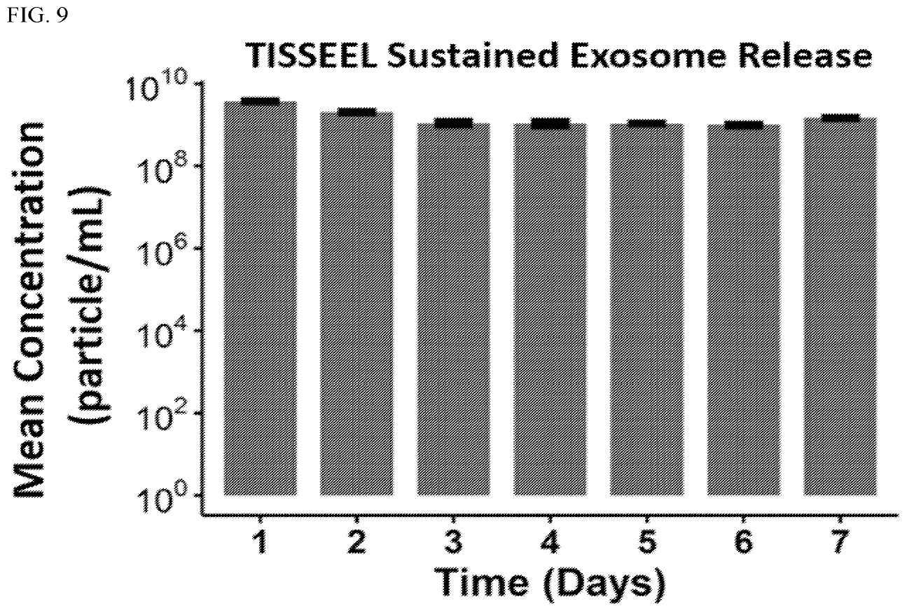

To assess effect of PEP on skeletal muscle in vivo, a rat volumetric muscle loss model was used in which a large muscle lesion was created to exceed the endogenous satellite stem cell capabilities for self-repair. Latissimus dorsi defects (8 mm) were created with a punch biopsy and treated with saline (sham; n=9), clinical-grade fibrin glue (TISSEEL, n=9), or TISSEEL reconstituted with 1×10¹² exosomes/ml (PEP, n=9) (FIG. 8A,B). Following reconstitution, PEP exosomes uniformly adhered to fibrin fibers as shown by scanning electron microscopy (FIG. 8C). Furthermore, TISSEEL in combination with 1×10¹² exosomes/ml resulted in the sustained release of exosomes (FIG. 9). Animals were treated with 2′-deoxy-5-ethynyluridine (EdU) as a thymidine analog to monitor cellular division and proliferation.

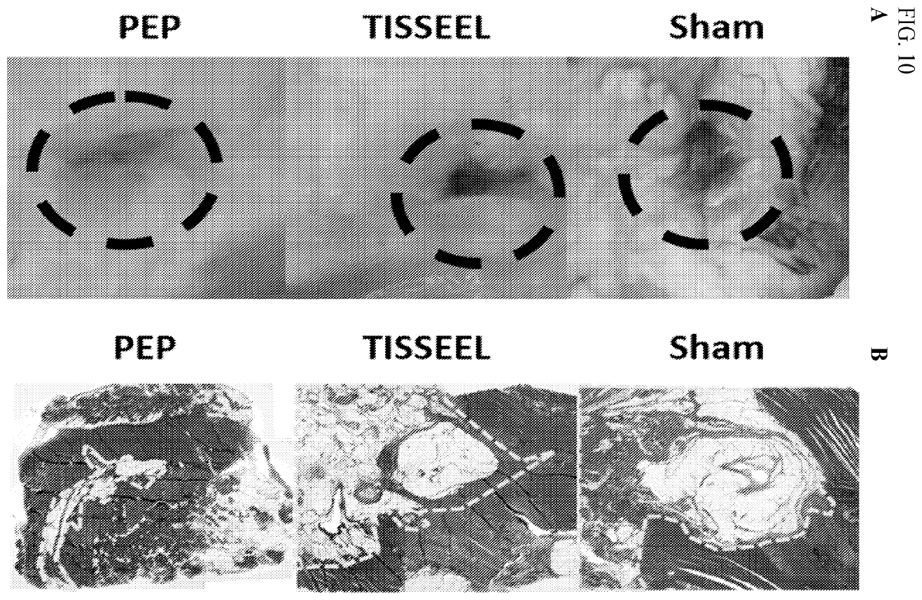

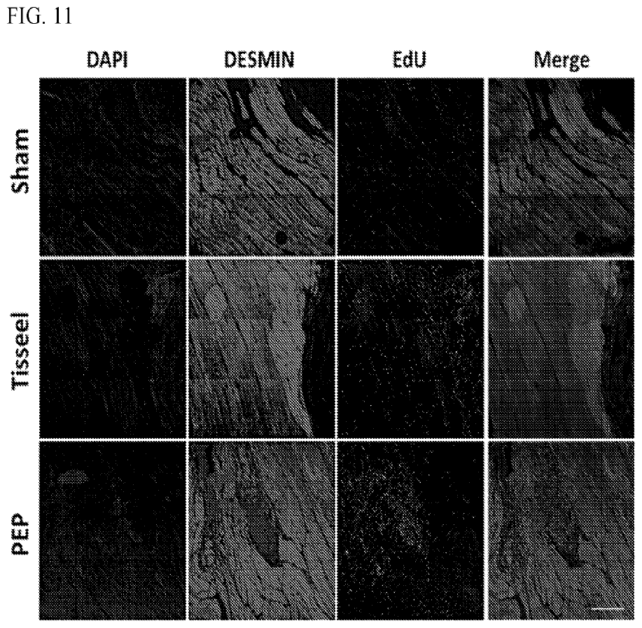

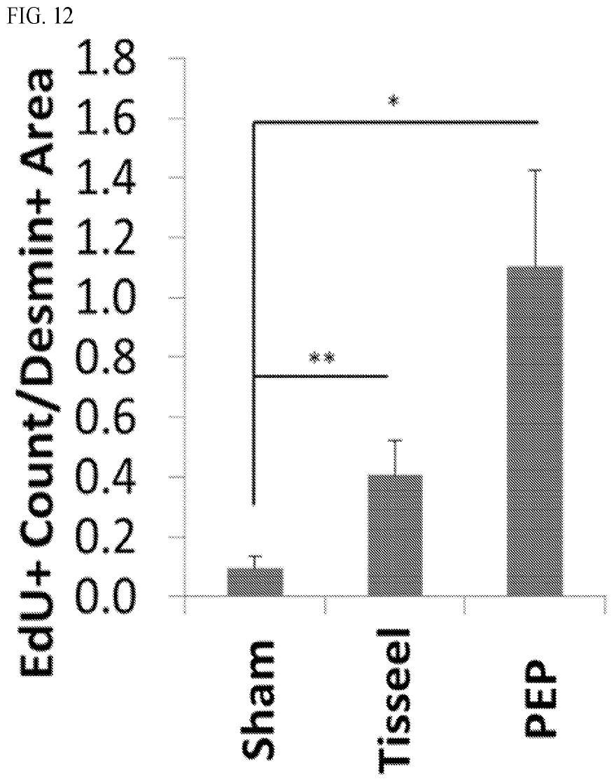

All animals survived to sacrifice at eight weeks post-treatment. Sham-treated animals had persistent muscle defects with fatty infiltration, while those repaired with TISSEEL or with TISSEEL and PEP had grossly healed defects (FIG. 10A). Histological characterization revealed that in contrast to the absence of tissue in sham, TISSEEL alone induced primarily a fatty infiltrate with inflammatory response. Conversely, defects treated with PEP had widespread skeletal muscle regrowth with resulting decrease in the area of the defect compared to both sham and TISSEEL-treated rats (FIG. 10A,B). Immunohistochemical evaluation revealed co-localization of EdU⁺ cells in desmin⁺ tissue areas suggesting de novo skeletal muscle regrowth at the injury site (FIG. 11). Significantly more EdU staining was observed in desmin positive cells per mm² compared to sham control (p<0.001), but not compared to TISSEEL treatment alone (p=0.24) (FIG. 12). These data suggest that PEP affects muscle regeneration at the site of injury.

Restoration of Urethral Pressures Following Urethral Sphincter Repair with PEP in a Novel Porcine SUI Model

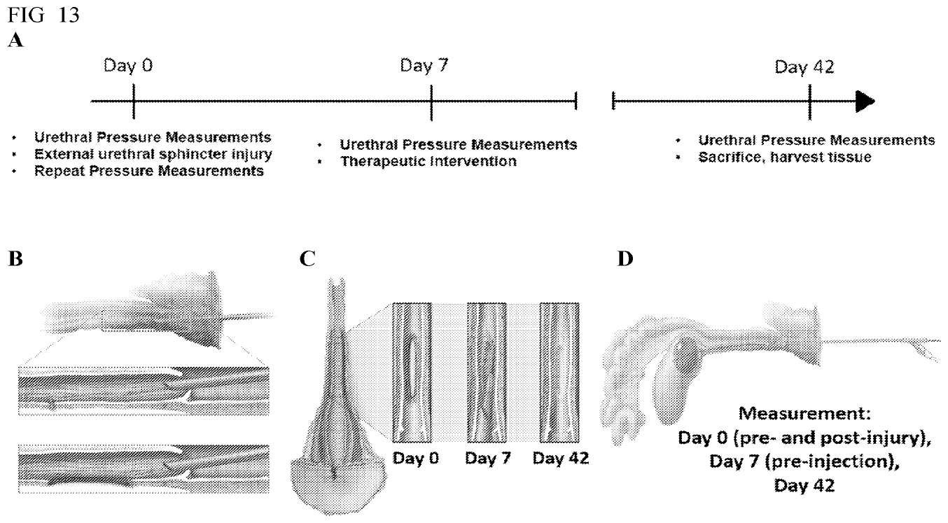

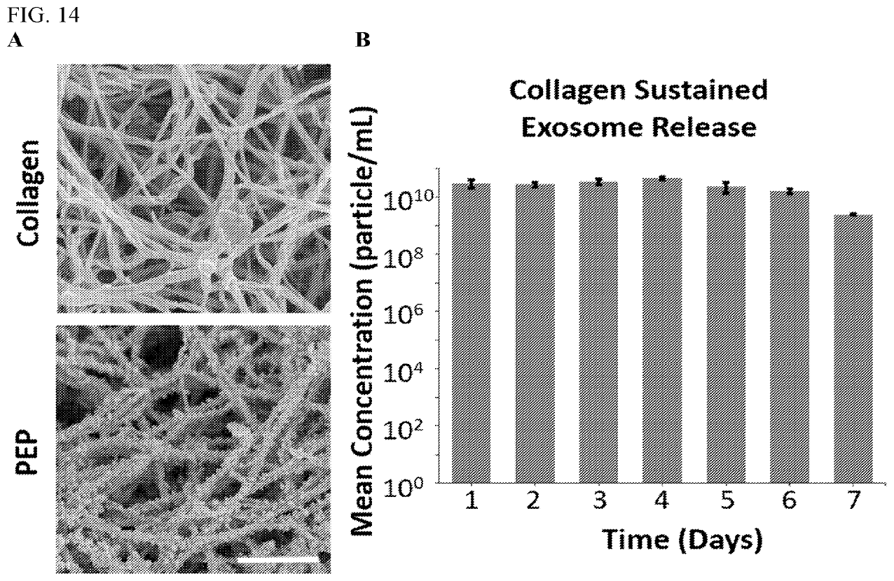

A porcine SUI model was used to assess whether skeletal muscle regenerated using PEP could integrate with surrounding uninjured muscle to restore pre-injury function of the urethral sphincter. This model was chosen due to anatomic similarities between human and pig urethral sphincters. A total of 10 female Yorkshire-Crossed Pigs weighing 70-80 kg were used (four controls, six experimental). Under aseptic conditions and general anesthesia, an approximately 2-cm long full thickness lesion, extending through the mucosa and urethral sphincter, was created cystoscopically at the 6 o'clock position starting 1 cm from the meatus and extending cephalad (FIG. 13B). The animals were allowed to convalesce for approximately seven days before returning to the operating room where a total of 5 ml of collagen or collagen with 1×10¹² PEP exosomes/ml was injected in approximately 0.5-cc aliquots over 10 injection points along the length of the previously created urethral sphincter defect (FIG. 13C). In contrast to TISSEEL, which polymerizes instantly, 5 mg/ml collagen hydrogel takes minutes to gel at 37° C., making it more suitable as the vehicle for injection through a cystoscope. PEP demonstrated similar binding affinity to collagen as it does to TISSEEL on SEM (FIG. 14A). Similarly, collagen in combination with 1×10¹² exosomes/mL resulted in the sustained release of exosomes (FIG. 14B). A urethral pressure profile was used to quantify sphincter function using a modified MEDSPIRA mCompass manometry pressure catheter. Pressures were recorded pre-injury and post-injury on day 0, pre-therapy on day 7, and prior to euthanasia 42 days post-therapy (FIG. 13D). Urethral pressures were recorded for 10 seconds, starting in the vagina and extending to the bladder neck in 1-cm increments (FIG. 13D). Three pressure readings from each 10 second recorded segment were averaged to produce a mean urethral pressure at that location. A dose of 5 mg/kg EdU was orally administered to all animals twice weekly throughout the study as a thymidine analog to monitor cellular division and proliferation.

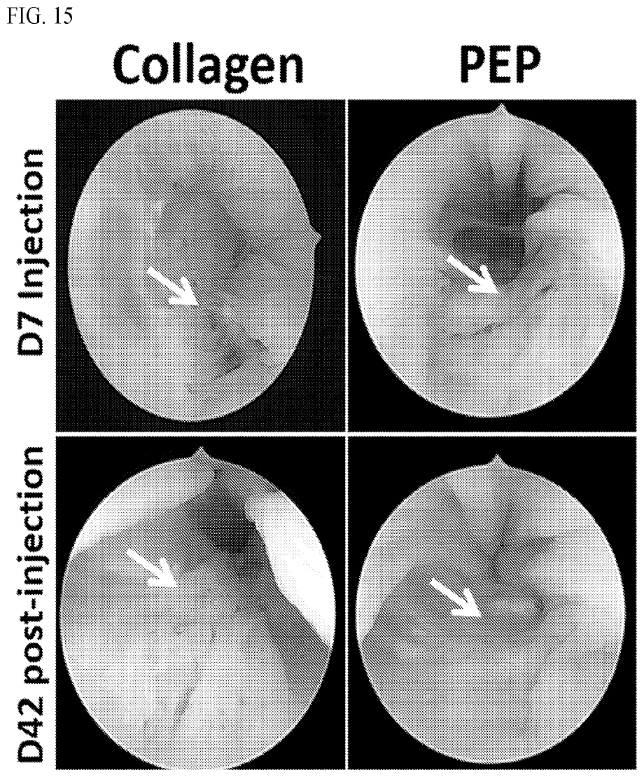

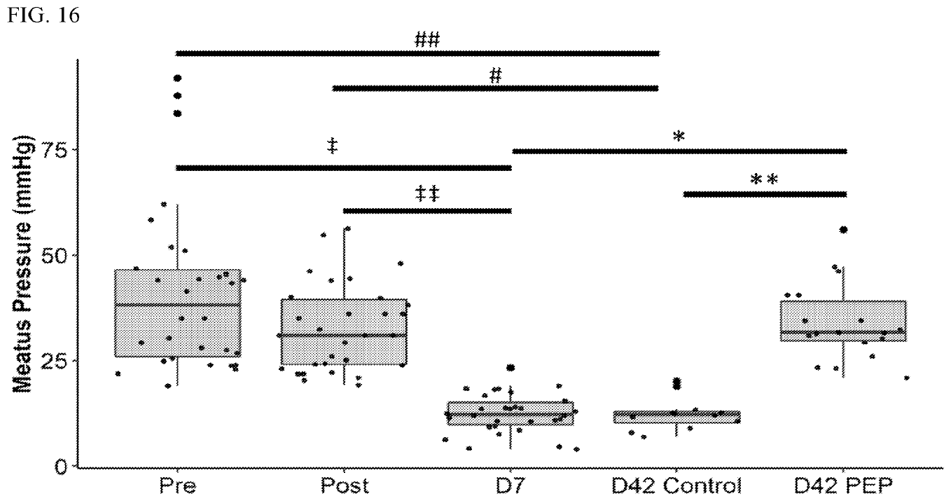

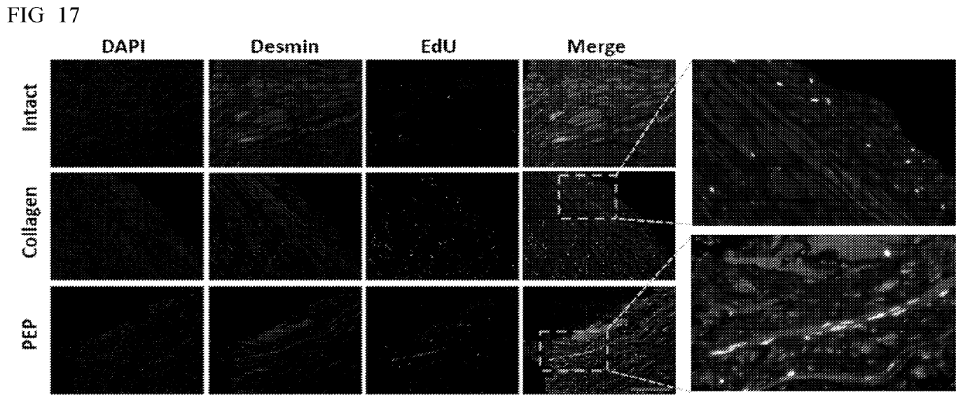

There were no deaths nor infections observed during the study. Two animals developed overt urinary incontinence between lesion creation and euthanasia, suggesting this approach provides a model with the desired impact on urethral sphincter function. Collagen-only treated animals had persistent muscle defects, while those repaired with collagen and PEP had grossly healed defects (FIG. 15). Although the mean urethral pressure minimally changed at day 0, it dropped significantly by day 7, quantitatively validating the model (FIG. 16). At day 42, animals treated with collagen alone had no significant improvement in sphincter function as measured by urethral pressure. In contrast, animals treated with PEP had significant restoration of urethral pressures versus day 7 pre-injection and day 42 collagen alone (FIG. 16).

### Histological Characterization of Urethral Sphincter Lesion Repair

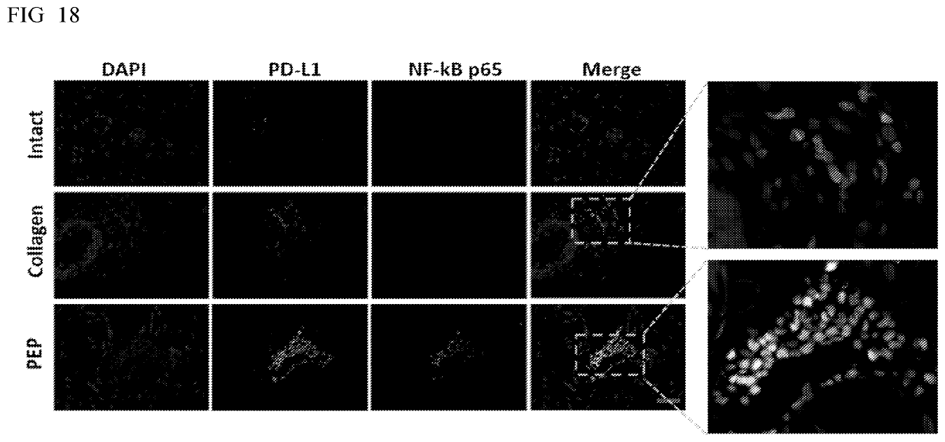

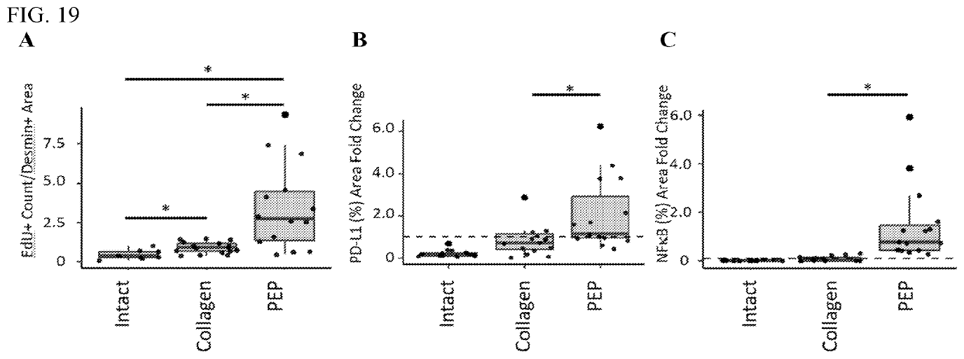

Representative sections from both the injury site and intact segments of the urethra were stained for desmin (a cytoskeletal intermediate filament found in sarcomeres of adult myoblasts) to identify skeletal muscle, EdU (thymidine analog used to monitor cellular division and proliferation) to identify newly synthesized cells, and DAPI to identify newly synthesized nuclei. While there was limited EdU/Desmin co-localization of the collagen-treated urethral lesion and intact urethral tissue (FIG. 17), abundant co-localization of desmin and EdU positive cells was seen at the injury site injected with PEP (FIG. 17). A magnified view of these areas revealed multinucleated cells staining positive for the above markers only in injury sites treated with PEP, suggesting de novo skeletal muscle cell regeneration with differentiation to multinucleated myotubes (FIG. 17, zoomed inset). Quantification by a blinded observer documented significantly more EdU positive cells per mm² in desmin⁺ areas in injury sites treated with PEP compared to collagen control and compared to the intact urethra (FIG. 19A).

### Injury Site NF-κB and PD-L1 Expression and Immune Response Elicited by PEP

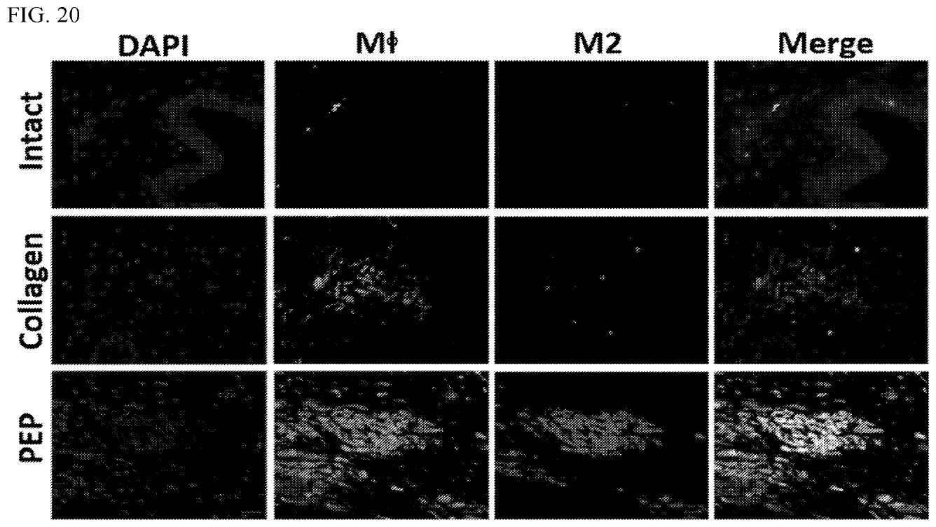

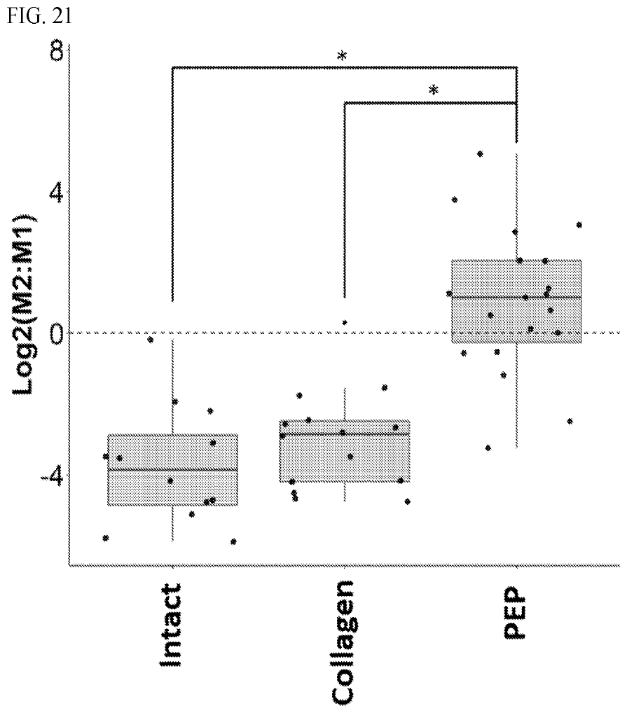

Expression and co-localization of PD-L1 and NF-κB was observed at significantly greater percentage in sections of injured urethra treated with PEP compared to sections on injured urethra treated only with collagen (FIG. 18). Expression of NF-κB and PD-L1 promotes M2 macrophage polarization, which promotes skeletal muscle regeneration. The intact urethra, lacking a triggering event, had limited macrophage staining for either M1 or M2 macrophages (FIG. 20, FIG. 21). The macrophage response to collagen treatment appeared restricted to a less pronounced undifferentiated macrophage staining with a paucity of M2 macrophages (FIG. 20, FIG. 21). In contrast, an abundance of M2 predominant macrophages was found only in those treated with PEP (FIG. 20). Quantification favored M2 polarization in PEP-treated cohorts, not observed with collagen alone (FIG. 21).

Thus, this disclosure describes the use of platelet-derived exosomes (PEP) to stimulate myoblast growth in vitro and in two distinct in vivo models. PEP provided a dose-dependent effect on myoblast proliferation, cell migration, and chemotaxis. Resveratrol inhibition of PEP-mediated myoblast proliferation implicated a critical role for NF-κB p65, a molecule detected within PEP. In vivo, treatment with PEP led to significant higher skeletal muscle repopulation in a VML latissimus dorsi model and improvement in urethral sphincter muscle function in a PEP-treated porcine model of SUI. EdU and desmin co-localization, sustained expression of NF-κB and PDL-1, and M2 macrophage polarization points to the in vivo activation of these established myoregenerative molecular cues within PEP-treated tissues. Collectively, these findings support the use of PEP, an exosome-based off-the-shelf acellular platform, for restoring skeletal muscle function.

The PEP exosome technology described here provides a potential solution to morbidity of muscle biopsy, high manufacturing cost, dose-to-dose variability, and hurdles associated with cell transport and handling that exist with using muscle cell precursor (MCP) therapies. PEP exosomes are scalable and stable at room-temperature stable as a lyophilized product. In addition, by activating local myocyte progenitors, PEP eliminates the need for exogenous myoblast transplantation to drive desired myoregenerative events.

Satellite cells are mononucleated skeletal muscle stem cells that reside closely opposed to mature myofibers in a dormant state until they become activated by muscle injury. Upon activation, there is cellular expansion to both replace the stem cell pool and repair damaged tissue. Cells destined for repair align along injured fibers and differentiate into myotubes that fuse with the surrounding uninjured muscle to heal the injury. Macrophages are pleotropic immune cells that, among other functions, are involved in muscle repair and regeneration. Specifically, an orchestrated shift from an early pro-inflammatory response to wound healing and repair responses is necessary for muscle regeneration following injury. The early pro-inflammatory response is mediated by M1 macrophages and is necessary for myofiber and wound debridement. Wound healing and repair involve direct interactions between M2 macrophages and satellites cells at the site of myofiber injury. Under local inflammatory conditions and during injury repair, myoblasts express a number of B7-related (CD80 and CD86) co-stimulatory and inhibitory molecules, including PD-L1, which is a potent immune-modulator detected in large quantities in PEP. Here, exogenous PD-L1, donated by PEP, drove a pro-regenerative transition from pro-inflammatory M1 to M2 macrophages in PEP-treated urethral sphincter sites.

This disclosure therefore describes compositions and methods for repairing damaged or injured skeletal muscle in a subject. Generally, the compositions include PEP and a pharmaceutically acceptable carrier. In a surgical setting, the PEP may be combined with a carrier that is suitable for repair of skeletal muscle such as, for example, a surgical glue, a tissue adhesive, and/or a supportive matrix (e.g., a collagen scaffold).

Thus, the method includes administering an effective amount of the composition to repair damaged skeletal muscle. In this aspect, an “effective amount” is an amount effective to induce expression of NK-κB, induce expression of PD-L1, and/or promote polarization toward M2 macrophages.

As used herein, a “subject” can be a human or any non-human animal. Exemplary non-human animal subjects include, but are not limited to, a livestock animal or a companion animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, reindeer, etc.), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

PEP may be formulated with a pharmaceutically acceptable carrier to form a pharmaceutical composition. As used herein, “carrier” includes any solvent, dispersion medium, vehicle, coating, diluent, antibacterial, and/or antifungal agent, isotonic agent, absorption delaying agent, buffer, hydrogel, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the PEP without causing any undesirable biological effects or interacting in a deleterious manner with any of the other components of the pharmaceutical composition in which it is contained. As noted above, in a surgical setting, exemplary suitable carriers include surgical glue, tissue adhesive, or a supportive matrix (e.g., a collagen scaffold).

In one or more embodiments, the PEP composition may include additional components, either in admixture with the PEP vesicles or loaded into at least a portion of the PEP vesicles. In one or more embodiments, the additional components can promote healing. In one or more embodiments, the additional component may include, for example, one or more biocompatible scaffold components such as, for example, collagen, fibrin, fibronectin, laminin, proteoglycan, hyaluronic acid, alginate, or other biocompatible scaffold components. In one or more embodiments, the additional component may include one or more pharmaceutically active ingredients. In one or more embodiments, the pharmaceutically active ingredient may be selected to, for example, decrease healing time, provide pain relief, or reduce the likelihood or severity of a complication (e.g., infection). A pharmaceutically active ingredient may include, for example, a steroid (e.g., estrogen), an analgesic, or an antibiotic. One or more additional component may be loaded into at least a portion of the PEP vesicles. The presence of certain components in a PEP preparation may be the result of, for example, expression of the component (e.g., a protein) by the donor cell from which the PEP was derived. Additionally or alternatively, one or more components may be loaded into at least a portion of the PEP vesicles either directly (loading of the component itself) or indirectly (e.g., recombinantly expressed proteins).

A pharmaceutical composition containing PEP may be formulated in a variety of forms adapted to a preferred route of administration. Thus, a pharmaceutical composition can be administered via known routes including, for example, oral, parenteral (e.g., intradermal, transcutaneous, subcutaneous, intramuscular, intravenous, intraperitoneal, etc.), or topical (e.g., application to nervous tissue exposed during surgery, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A pharmaceutical composition also can be administered via a sustained or delayed release.

Thus, a pharmaceutical composition may be provided in any suitable form including but not limited to a solution, a suspension, an emulsion, a spray, an aerosol, or any form of mixture. The pharmaceutical composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the PEP into association with a carrier that constitutes one or more accessory ingredients. In general, a formulation may be prepared by uniformly and/or intimately bringing the PEP into association with a liquid carrier, a finely divided solid carrier, or both, and then, if necessary, shaping the product into the desired formulations.

The amount of PEP administered can vary depending on various factors including, but not limited to, the content and/or source of the PEP being administered, the weight, physical condition, and/or age of the subject, and/or the route of administration. Thus, the absolute weight of PEP included in a given unit dosage form can vary widely, and depends upon factors such as the species, age, weight, and physical condition of the subject, and/or the method of administration. Accordingly, it is not practical to set forth generally the amount that constitutes an amount of PEP effective for all possible applications. Those of ordinary skill in the art, however, can readily determine the appropriate amount with due consideration of such factors.

In one or more embodiments, a dose of PEP can be measured in terms of the PEP exosomes delivered in a dose. Thus, in one or more embodiments, the method can include administering sufficient PEP to provide a dose of, for example, from about 1×10⁶ PEP exosomes to about 1×10¹⁵ PEP exosomes to the subject, although in one or more embodiments the methods may be performed by administering PEP in a dose outside this range.

In one or more embodiments, therefore, the method can include administering sufficient PEP to provide a minimum dose of at least 1×10⁶ PEP exosomes, at least 1×10⁷ PEP exosomes, at least 1×10⁸ PEP exosomes, at least 1×10⁹ PEP exosomes, at least 1×10¹⁰ PEP exosomes, at least 1×10¹¹ PEP exosomes, at least 2×10¹¹ PEP exosomes, at least 3×10¹¹ PEP exosomes, at least 4×10¹¹ PEP exosomes, at least 5×10¹¹ PEP exosomes, at least 6×10¹¹ PEP exosomes, at least 7×10¹¹ PEP exosomes, at least 8×10¹¹ PEP exosomes, at least 9×10¹¹ PEP exosomes, at least 1×10¹² PEP exosomes, 2×10¹² PEP exosomes, at least 3×10¹² PEP exosomes, at least 4×10¹² PEP exosomes, or at least 5×10¹² PEP exosomes, at least 1×10¹³ PEP exosomes, or at least 1×10¹⁴ PEP exosomes.

In one or more embodiments, the method can include administering sufficient PEP to provide a maximum dose of no more than 1×10¹⁵ PEP exosomes, no more than 1×10¹⁴ PEP exosomes, no more than 1×10¹³ PEP exosomes, no more than 1×10¹² PEP exosomes, no more than 1×10¹¹ PEP exosomes, or no more than 1×10¹⁰ PEP exosomes.

In one or more embodiments, the method can include administering sufficient PEP to provide a dose characterized by a range having endpoints defined by any a minimum dose identified above and any maximum dose that is greater than the minimum dose. For example, in one or more embodiments, the method can include administering sufficient PEP to provide a dose of from 1×10¹¹ to 1×10¹³ PEP exosomes such as, for example, a dose of from 1×10¹¹ to 5×10¹² PEP exosomes, a dose of from 1×10¹² to 1×10¹³ PEP exosomes, or a dose of from 5×10¹² to 1×10¹³ PEP exosomes. In one or more certain embodiments, the method can include administering sufficient PEP to provide a dose that is equal to any minimum dose or any maximum dose listed above. Thus, for example, the method can involve administering a dose of 1×10¹⁰ PEP exosomes, 1×10¹¹ PEP exosomes, 5×10¹¹ PEP exosomes, 1×10¹² PEP exosomes, 5×10¹² PEP exosomes, 1×10¹³ PEP exosomes, or 1×10¹⁴ PEP exosomes.

Alternatively, a dose of PEP can be measured in terms of the concentration of PEP upon reconstitution from a lyophilized state. Thus, in one or more embodiments, the methods can include administering PEP to a subject at a dose of, for example, from about a 0.01% solution to a 100% solution to the subject, although in one or more embodiments the methods may be performed by administering PEP in a dose outside this range. As used herein, a 100% solution of PEP refers to one vial of PEP (approximately 2×10¹¹ exosomes or 75 mg) solubilized in 1 ml of a liquid or gel carrier (e.g., water, phosphate buffered saline, serum free culture media, surgical glue, tissue adhesive, etc.). For comparison, a dose of 0.01% PEP is roughly equivalent to a standard dose of exosomes prepared using conventional methods of obtaining exosomes such as exosome isolation from cells in vitro using standard cell conditioned media.

In one or more embodiments, therefore, the method can include administering sufficient PEP to provide a minimum dose of at least 0.01%, at least 0.05%, at least 0.1%, at least 0.25%, at least 0.5%, at least 1.0%, at least 2.0%, at least 3.0%, at least 4.0%, at least 5.0%, at least 6.0%, at least 7.0%, at least 8.0%, at least 9.0%, at least 10%, at least 15%, at least 20%, at least 25%, at least 30%, at least 35%, at least 40%, at least 45%, at least 50%, at least 60%, or at least 70%.

In one or more embodiments, the method can include administering sufficient PEP to provide a maximum dose of no more than 100%, no more than 90%, no more than 80%, no more than 70%, no more than 60%, no more than 50%, no more than 40%, no more than 30%, no more than 20%, no more than 10%, no more than 9.0%, no more than 8.0%, no more than 7.0%, no more than 6.0%, no more than 5.0%, no more than 4.0%, no more than 3.0%, no more than 2.0%, no more than 1.0%, no more than 0.9%, no more than 0.8%, no more than 0.7%, no more than 0.6%, no more than 0.5%, no more than 0.4%, no more than 0.3%, no more than 0.2%, or no more than 0.1%.

In one or more embodiments, the method can include administering sufficient PEP to provide a dose characterized by a range having endpoints defined by any a minimum dose identified above and any maximum dose that is greater than the minimum dose. For example, in one or more embodiments, the method can include administering sufficient PEP to provide a dose of from 1% to 50% such as, for example, a dose of from 5% to 20%. In one or more certain embodiments, the method can include administering sufficient PEP to provide a dose that is equal to any minimum dose or any maximum dose listed above. Thus, for example, the method can involve administering a dose of 0.05%, 0.25%, 1.0%, 2.0%, 5.0%, 20%, 25%, 50%, 80%, or 100%.

A single dose may be administered all at once, continuously for a prescribed period of time, or in multiple discrete administrations. When multiple administrations are used, the amount of each administration may be the same or different. For example, a prescribed daily dose of may be administered as a single dose, continuously over 24 hours, as two administrations, which may be equal or unequal. When multiple administrations are used to deliver a single dose, the interval between administrations may be the same or different. In one or more certain embodiments, PEP may be administered as a once-off administration such as, for example, during a surgical procedure.

In one or more certain embodiments in which multiple administrations of the PEP composition are administered to the subject, the PEP composition may be administered as needed to repair or heal injured or damaged skeletal muscle to the desired degree. Alternatively, the PEP composition may be administered twice, three times, four times, five times, six times, seven times, eight times, nine times, or at least ten times. The interval between administrations can be a minimum of at least one day such as, for example, at least three days, at least five days, at least seven days, at least ten days, at least 14 days, or at least 21 days. The interval between administrations can be a maximum of no more than six months such as, for example, no more than three months, no more than two months, no more than one month, no more than 21 days, or no more than 14 days.

In one or more embodiments, the method can include multiple administrations of PEP to a subject at an interval (for two administrations) or intervals (for more than two administrations) characterized by a range having endpoints defined by any minimum interval identified above and any maximum interval that is greater than the minimum interval. For example, in one or more embodiments, the method can include multiple administrations of PEP at an interval or intervals of from one day to six months such as, for example, from three days to ten days. In one or more certain embodiments, the method can include multiple administrations of PEP at an interval of that is equal to any minimum interval or any maximum interval listed above. Thus, for example, the method can involve multiple administrations of PEP at an interval of three days, five days, seven days, ten days, 14 days, 21 days, one month, two months, three months, or six months.

In one or more embodiments, the methods can include administering a cocktail of PEP that is prepared from a variety of cell types, each cell type having a unique myoregenerative profile—e.g., protein composition and/or gene expression. In this way, the PEP composition can provide a broader spectrum of myoregenerative activity than if the PEP composition is prepared from a single cell type.

In the preceding description and following claims, the term “and/or” means one or all of the listed elements or a combination of any two or more of the listed elements; the terms “comprises,” “comprising,” and variations thereof are to be construed as open ended i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” “one or more certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

### EXEMPLARY EMBODIMENTS

Embodiment 1 is a method of treating injured skeletal muscle tissue, the method comprising applying a therapeutic composition to injured skeletal muscle tissue, the therapeutic composition comprising:
a purified exosome product (PEP); and
a pharmaceutically acceptable carrier.

Embodiment 2 is the method of Embodiment 1, wherein the PEP comprises spherical or spheroid exosomes having a diameter no greater than 300 nm.

Embodiment 3 is the method of Embodiment 1, wherein the PEP comprises spherical or spheroid exosomes having a mean diameter of 110 nm±90 nm.

Embodiment 4 is the method of Embodiment 3, wherein the PEP comprises spherical or spheroid exosomes having a mean diameter of 110 nm±50 nm.

Embodiment 5 is the method of Embodiment 4, wherein the PEP comprises spherical or spheroid exosomes having a mean diameter of 110 nm±30 nm.

Embodiment 6 is the method of any preceding Embodiment, wherein the PEP comprises:
from 1% to 20% CD63⁻ exosomes; and
from 80% to 99% CD63⁺ exosomes.

Embodiment 7 is the method of any one of Embodiments 1-5, wherein the PEP comprises at least 50% CD63⁻ exosomes.

Embodiment 8 is the method of any preceding Embodiment, wherein the PEP comprises from 1×10¹¹ PEP exosomes to 1×10¹³ PEP exosomes.

Embodiment 9 is the method of Embodiment 8, wherein the PEP comprises from 1×10¹² PEP exosomes to 1×10¹³ PEP exosomes.

Embodiment 10 is the method of any preceding Embodiment, wherein the therapeutic composition further comprises a supportive matrix.

Embodiment 11 is the method of Embodiment 10, wherein the supportive matrix comprises a collagen scaffold.

Embodiment 12 is the method of any preceding Embodiment, wherein the therapeutic composition further comprises a tissue sealant or fibrin glue.

Embodiment 13 is the method of any preceding Embodiment, wherein the therapeutic composition forms a hydrogel after being applied to the injured skeletal muscle.

Embodiment 14 is the method of Embodiment 13, wherein the therapeutic composition forms a hydrogel when incubated at a temperature of 35° C. to 40° C.

Embodiment 15 is the method of Embodiment 13 or Embodiment 14, wherein the therapeutic composition forms a hydrogel at least one minute after being applied to skeletal muscle.

Embodiment 16 is the method of any preceding Embodiment, wherein the therapeutic composition is applied in an amount effective to increase expression of NF-κB in the injured skeletal muscle tissue compared to injured skeletal muscle tissue treated without the therapeutic composition.

Embodiment 17 is the method of any preceding Embodiment, wherein the therapeutic composition is applied in an amount effective to increase expression of PD-L1 in the injured skeletal muscle tissue compared to injured skeletal muscle tissue treated without the therapeutic composition.

Embodiment 18 is the method of any preceding Embodiment, wherein the therapeutic composition is applied in an amount effective to increase polarization toward M2 macrophages in the injured skeletal muscle tissue compared to injured skeletal muscle tissue treated without the therapeutic composition.

Embodiment 19 is the method of any preceding Embodiment, wherein the injured skeletal muscle tissue comprises urethral sphincter muscle.

Embodiment 20 is the method of any preceding Embodiment, wherein at least a portion of the PEP exosomes comprise PD-L1, NF-κB, or both PD-L1 and NF-κB.

Embodiment 21 is the method of any preceding Embodiment, wherein at least a portion of the PEP exosomes comprise CD63, CD9, flotillin, or any combination of two or more of the foregoing.

Embodiment 22 is the method of any preceding Embodiment, wherein the therapeutic composition is applied in an amount effective to increase cellular proliferation in the injured skeletal muscle tissue compared to injured skeletal muscle tissue treated without the therapeutic composition.

### EXAMPLES

The present invention is illustrated by the following examples. It is to be understood that the particular examples, materials, amounts, and procedures are to be interpreted broadly in accordance with the scope and spirit of the invention as set forth herein.

### Study Design

The aim of this study was to assess the myoregenerative potential of the Purified Exosome Product (PEP) acellular platform. A urethral sphincter model was used to evaluate the potential use of PEP as a non-mesh-based alternative to surgical treatment for stress urinary incontinence (SUI). The proliferative, migratory, and chemotaxis responses of human skeletal muscle myoblasts to PEP were evaluated. Proliferation assays following pretreatment of myoblasts with increasing concentrations of resveratrol led to inhibition of myoblast growth and suggested that an exosome dependent NF-κB signaling pathway may be required for satellite cell activation and differentiation.

Second, a volumetric muscle loss (VML) rat model was created to determine the in vivo efficacy of muscle regeneration and repair by PEP. Animals were randomized to saline, TISSEEL (Baxter International, Inc., Deerfield, IL), or TISSEEL reconstituted with 1×10¹² PEP exosomes/mL and the repair response characterized with Masson's trichrome and immunostaining against EdU and desmin.

Third, a porcine stress urinary incontinence (SUI) model was used to evaluate PEP for treating urethral sphincter defects. The created sphincter defect was sufficiently large to impair function and cause a significant decrease in urethral pressures. Animals underwent surgical repair of the urethral sphincter lesions with injection of collagen or collagen with 1×10¹² PEP exosomes/mL. Group sizes were chosen based on IACUC protocols and previous experience with large animal experiments. Investigators were blinded when collecting and analyzing data but were not blinded to treatment group during injection of active product.

### Characterization and Quantification of Purified Exosome Product

Purified Exosome Product (PEP; Rion LLC, Rochester, MN) is purified from the conditioned medium of apheresis purified platelets as previously described (Kisby et al., 2021, Female Pelvic Med Reconstr Surg. 27(10):609-615; Qi et al., 2020, J Orthop Res. 38(8):1845-1855; Shi et al., 2021, J Orthoped Res. 39(8):1825-1837; International Patent Publication No. WO 2019/118817; U.S. Pat. No. 10,596,123). Briefly, conditioned medium derived from purified platelets underwent serial filtration to remove any cytological material and to yield extracellular vesicles in the range of 50 nm to 200 nm. Further processing was achieved through staged filtration and centrifugation to ensure elimination of non-EV material. The extracellular vesicles were lyophilized to yield a dry powder containing approximately 5×10¹² vesicles per vial. Different exosome concentrations were created by reconstituting the lyophilized exosome in saline or in the described suspension media. Nanoparticle tracking analysis was conducted on a nanoparticle tracking analyzer (NanoSight NS3000, NanoSight, Ltd., Salisbury, United Kingdom) to confirm the presence of exosomes, characterize the particle size distribution, and quantify exosome concentration. Transmission electron microscopy (TEM) was conducted at a Mayo Clinic Microscopy and Cell Analysis Core Facility on a JEM-1400 Series 120 kV microscope (JEOL) with an acceleration voltage of 80 kV and an indicated magnification of 60 kx. TEM was conducted with PEP in solution, TISSEEL or collagen, and TISSEEL or collagen reconstituted with 1×10¹² PEP exosomes/mL.

### Western Blotting Technique and Quantification

Western blotting for expression of exosome markers was conducted in two different manners: traditional and Li-Cor. In the traditional method, equal amounts of protein were loaded into each lane and transferred to a PVDF membrane. The membranes were blocked in 5% nonfat dried milk diluted in TBST and incubated with primary antibodies against CD63, CD9, and Flotillin (Table 1). HIRP-conjugated secondary antibodies were used to incubate the membranes and were developed with enhanced chemiluminescence (AERSHAM, GE Healthcare, Chicago, TL). Loading was standardized to a GAPDH loading control. In the Li-Cor digital fluorescence method (Li-Cor Biosciences, Inc., Lincoln, NE), equal amounts of protein were loaded into each lane and transferred to a nitrocellulose membrane. The membranes were blocked in Li-Cor TBS Blocking Buffer and incubated with primary antibodies against NF-κB p65, and PD-L1. Secondary antibodies at 680 nm and 800 nm were used to fluorescently image the membranes using an imaging system (Odyssey CLx, Li-Cor Biosciences, Inc., Lincoln, NE). Digital fluorescent intensity was determined by IMAGE STUDIO software (Li-Cor Biosciences, Inc., Lincoln, NE) and loading was standardized by total protein stain (Li-Cor Biosciences, Inc., Lincoln, NE) for whole lane protein digital fluorescence and analyzed by EMPIRIA STUDIO software (Li-Cor Biosciences, Inc., Lincoln, NE).

TABLE 1
Primary antibodies used for immunofluorescence

| 1° Antibody | Company | Catalog # | Species | Dilution |
|---|---|---|---|---|
| Desmin | Abcam plc, | ab32362 | Rabbit | 1:250 |
| | Cambridge, | | | |
| | United Kingdom | | | |
| NF-ϰB p65 | Santa Cruz | Sc-8008 | Mouse | IHC: 1:200 |
| | Biotechnology, | | | WB: 1:1000 |
| | Inc., Dallas, TX | | | |
| PD-L1 | Cell Signaling | 13684S | Rabbit | IHC: 1:200 |
| | Technology, | | | WB: 1:1000 |
| | Inc., Danvers, MA | | | |
| Mϕ | LifeSpan | LS-B9966 | Mouse | 1:100 |
| | BioSciences, | | | |
| | Inc., Seattle, WA | | | |
| CD 163 | Abcam plc, | ab87099 | Rabbit | 1:200 |
| | Cambridge, | | | |
| | United Kingdom | | | |
| CD63 | Abcam plc, | ab271296 | Mouse | 1:1000 |
| | Cambridge, | | | |
| | United Kingdom | | | |
| CD9 | Cell Signaling | D801A | Rabbit | 1:1000 |
| | Technology, | | | |
| | Inc., Danvers, MA | | | |
| Flotillin-1 | Cell Signaling | 3253S | Rabbit | 1:1000 |
| | Technology, | | | |
| | Inc., Danvers, MA | | | |
| Pax7 | Thermo Fisher | PA1-117 | Rabbit | 1:100 |
| | Scientific, | | | |
| | Inc., Waltham, MA | | | |
| MyoD | Thermo Fisher | MA1-41017 | Mouse | 1:200 |
| (5.8A) | Scientific, | | | |
| | Inc., Waltham, MA | | | |
| Heavy Chain | Abcam plc, | ab50967 | Mouse | 1:100 |
| Cardiac | Cambridge, | | | |
| Myosin | United Kingdom | | | |

### Atomic Force Microscope (AFM) and Single-Particle Interferometric Reflectance Imaging Sensing (SP-IRIS) Analysis

Contact-mode AFM with silicon nitride NP-S tips (spring constant, 0.58 Newton/meter) was performed on a Nanoscope III controller (Digital Instruments). Exosomes either purified through traditional ultracentrifugation or through methodology described above (PEP) were fixed in situ, rinsed with ultrapure water (18 MΩ-cm) and dried. Images were obtained using linear scanning frequencies (5-15 Hz) to generate 512×512-pixel AFM images. Three-dimensional topographical images were generated and quantified using Nanoscope software.

Two lots, in triplicate, of lyophilized PEP were reconstituted in 1 mL Incubation Solution I (NanoView Biosciences) and further diluted 1,000-fold in the same solution prior to incubating 50 μL on chip, for 16 hours according to the manufacturer's protocol for the ExoView human tetraspanin plasma kit (NanoView Biosciences; EV-TETRA-P). ExoView tetraspanin plasma chips are spotted with capture antibodies against CD41a and the mouse IgG1,κ isotype control in triplicate. The chips were then washed in an automated chip washer and incubated with conjugated antibodies for fluorescent labeling of the captured PEP (CF488a anti-CD9, NanoView Biosciences) for 1 hour. After labeling, the chips were washed and dried in the automated chip washer and placed in the reader for analysis. All data were gathered using an ExoView R100 reader equipped with ExoView Scanner 3.0 software and analyzed using ExoView Analyzer 3.0. All data reported was obtained and analyzed in blinded fashion adhering to CGMP quality-controlled standards.

### Characterization of the Effect of PEP on Human Skeletal Muscle Myoblasts

Human Skeletal Muscle Myoblasts (HSMM, Lonza Group AG, Basel, Switzerland, cat. #CC-2580) growth, migratory properties, and chemotaxis properties were assessed with cell culture with basal media (SkBM-2 Skeletal Muscle Cell Growth Basal Media, Lonza Group AG, Basel, Switzerland, cat. #CC-3246), basal media supplemented with 10% FBS (SkGM-2 SingleQuots Supplements and Growth Factors, Lonza Group AG, Basel, Switzerland, cat. #CC-3244) or basal media with increasing concentrations of PEP ranging from 1.25×10¹¹ PEP exosomes/mL to 5×10¹¹ PEP exosomes/mL was assessed using a live cell analyzer (INCUCYTE, Essen BioScience, Inc., Ann Arbor, MI). Data was generated from the live cell analysis by 5 acquiring phase images (10× lens; 4 images/well in 96 well plate with 12 wells/condition).

Chemotactic properties of PEP were assessed using chemotaxis module for the live cell analyzer (INCUCYTE, Essen BioScience, Inc., Ann Arbor, MI). HSMM in media were plated on the top chamber and chemotaxis was assessed by placing basal media, media supplemented with 10% FBS, or media with increasing concentrations of PEP ranging from 1.25×10¹¹ PEP exosomes/mL to 5×10¹¹ PEP exosomes/mL in the bottom chamber. Images were collected from both the top and the bottom chambers. Total phase area in the top well was normalized to initial top value with a decrease in cell area in the top chamber indicating enhanced cell migration through the pore and improved chemotaxis. Graph was plotted using the chemotaxis module for the live cell analyzer (INCUCYTE, Essen BioScience, Inc., Ann Arbor, MI) by acquiring wide angle phase images (10× lens; 1 image/well in 96 well plate with 12 wells/condition).

HSMM migration was assessed using a scratch wound assay. Graph is plotted at different time points with basal media, media supplemented with 10% FBS, and media with increasing concentrations of PEP ranging from 1.25×10¹¹ PEP exosomes/mL to 5×10¹¹ PEP exosomes/mL. Data is generated from the live cell analyzer (INCUCYTE, Essen BioScience, Inc., Ann Arbor, MI) by acquiring phase images (10× lens; 1 image/well in 96-well plate with 12 wells/condition).

### Resveratrol Inhibition of Human Skeletal Muscle Myoblast Proliferation

The mechanism of PEP-induced HSMM proliferation was interrogated using resveratrol (InvivoGen, San Diego, CA), an NF-κB inhibitor, in culture grown with 2.5×10¹¹ PEP exosomes/mL. The concentration of PEP exosomes was chosen both because of its optimal impact on the growth of cultured HSMMs and to match the empirically determined timed release PEP concentration from TISSEEL (Baxter International, Inc., Deerfield, IL) reconstituted with 1×10¹² PEP exosomes/mL. HSMM were plated on a 96-well plate at 10,000 cells/well overnight. Resveratrol was incubated at concentrations ranging from 50 μM to 500 μM for an hour before adding 2.5×10¹¹ PEP exosomes/mL to each well. Data is generated from live cell proliferation analysis (INCUCYTE, Essen BioScience, Inc., Ann Arbor, MI) by acquiring phase images (10× lens; five images/well in 96-well plate with three wells/condition). Western blotting for expression of NF-κB p65 at 72 hours post inhibition was quantified and normalized (IMAGE STUDIO software, LI-COR Biosciences, Inc., Lincoln, NE) using a total protein stain.

### Exosome Release Assay

For the TISSEEL release assay, a TISSEEL kit (Baxter International, Inc., Deerfield, IL) was prepared by adding 1 ml of fibrinolysis inhibitor to sealer protein concentrate (Baxter International, Inc., Deerfield, IL) and adding 1 mL of CaCl₂) solution to thrombin 500. Both solutions were incubated for 20 minutes at 37° C. One vial of PEP was resuspended with 400 μl heparin and 1.25 ml deionized water. The entire PEP solution was added to the fibrinolysis inhibitor/sealer protein concentrate solution. This solution was combined in each well of a 12-well plate in a 2:1 ratio with the CaCl₂)/thrombin 500 solution for a total volume of 990 μl per well.

For the collagen release assay, one vial of PEP was reconstituted in 2.5 ml of sterile water and filtered through a 0.22 μm filter. 500 μl PEP was combined with 500 μl of 6 mg/mL collagen (Collagen Solutions plc, Glasgow, United Kingdom). NaOH was added to a final concentration of 0.02 M. The entire 1 mL solution was added to a single well of a 12-well plate and incubated at 37° C. until solid. The final concentration of PEP for both assays in each well was 1×10¹² exosomes/mL. One mL of serum-free Dulbecco's Modified Eagle Medium 1× (DMEM) was added to each well. All media was collected daily and replaced with another 1 mL of serum free DMEM for the time course of the experiment.

### Repair of a Rat Latissimus Dorsi Volumetric Muscle Loss Model

After an acclimation period of 48 to 72 hours, 27 two-to-four-month-old male Lewis rats (Envigo, Indianapolis, IN) were housed and handled according to the Mayo Clinic Institutional Animal Care and Use Committee (IACUC) regulations (Mayo Clinic, Rochester, MN). Animals were clipped and anesthetized via isoflurane inhalation prior to surgery and maintained by intravenous fluids and isoflurane for the duration of the procedure. After the skin was prepped with betadine, a 3-cm longitudinal incision was made above the scapula, the skin mobilized with Metzembaum scissors and the latissimus dorsi (LD) muscle exposed. An 8-mm punch biopsy was used to create the LD volumetric muscle loss; taking care to not exceed ˜2 mm depth to avoid entering the chest cavity. The defect was filled with saline, tissue sealant (TISSEEL; Baxter International, Deerfield, IL), or TISSEEL reconstituted with 1×10¹² exosomes/mL PEP. The skin was closed with interrupted 2.0 VICRYL suture (Ethicon, Inc., Raritan, NJ). 2′-deoxy-5-ethynyluridine (EdU) was administered intraperitoneally at a dosage of 50 mg/kg once per week as a means of tracking cell proliferation. Animals were sacrificed at six weeks.

### Directed Urethral Sphincter Defect Porcine Model to Assess the Efficacy of PEP Induced Sphincter Muscle Regeneration

After an acclimation period of 48 to 72 hours, 10 female domestic Yorkshire-Crossed pigs, weighing 70-80 kg, underwent two survival surgeries and one terminal surgery. All animals underwent the outlined procedures, though their injected intervention differed as described below.

Anesthesia for survival surgery #1 was induced with TELAZOL (Zoetis, Parsippany, NJ; 5 mg/kg), xylazine (1-2 mg/kg) and maintained with isoflurane-vaporizer (1.5% to 3%). After obtaining general anesthesia, the pig was prepared and draped in a sterile fashion. Ceftiofur (5 mg/kg) was given intramuscularly for antibiotic prophylaxis. Cystoscopy was performed, the urethra identified, and a whistle tip stent placed. Using cystoscopic guidance the catheter (Medspira LLC, Minneapolis, MN) was placed in the urethra. The stent was removed, bladder balloon inflated to 30 cubic centimeters (cc), and pressures were obtained starting in the vagina and extending to the bladder neck in 1 cm increments. Pressures were collected for 10 seconds at each point and 3 pressures along a given 10-second recording averaged. Pressure catheter balloon was deflated, catheter removed, and stent used to re-identify the urethra. Using a Collin's knife, a 2-3 cm long, full thickness trans-mural defect involving the urethra wall and underlying sphincter muscle was created approximately 1 cm cephalad of the meatus (cut setting at 70 watts). Light hemostasis was obtained with Collin's knife (coagulation setting at 30 watts). A Foley catheter was placed for 48-72 hours to prevent urinary retention. Position was confirmed cystoscopically and the catheter secured with a 2.0 PROLENE suture (Ethicon, Inc., Raritan, NJ). The animals were allowed to heal for approximately seven days and returned to the operating room for the intervention procedure. Buprenorphine (0.03 mg/kg) was given every 6-8 hours for analgesia.

Anesthesia for survival surgery #2 was obtained as described above for surgery #1. Cystoscopy was used to identify the urethra and the catheter used to measure pressures as previously described. After pressure readings were collected, cystoscopy was used to inject the length of previously created defect with collagen and 1×10¹² exosomes/mL PEP (n=4) or collagen alone (n=2). A 1×10¹² exosomes/mL PEP gel was created by reconstituting PEP (Rion LLC, Rochester, MN) in 1 mL of sterile water (Hospira, Inc., Lake Forest, IL) and 4 mL of clinical grade type I bovine collagen (5 mg/mL; Collagen Solutions, Glasgow, UK). Collagen was made by adding 1 ml of sterile water to 4 ml of clinical grade type I bovine collagen (5 mg/mL; Collagen Solutions, Glasgow, UK). A total of 5 ml was injected in ˜0.5 ml aliquots along the length of the lesion using a 21-gauge needle (INJETAK, Laborie Medical Technologies, Corp., Portsmouth, NH). A prototype PEP delivery device was also used to deliver 1×10¹² exosomes/mL PEP+ Collagen (n=2) and collagen alone (n=2). This device allows for the deployment of two needles at the 4 o'clock and 8 o'clock positions at specific points along the length of the urethra and is being developed to avoid the need for cystoscopy or general anesthesia. The device, specially modified for the porcine anatomy, was placed in the urethra, the needles deployed, 1 cc injected, and the needles retracted. This process was repeated five times starting approximately 1 cm from urethra meatus and extending cephalad. Position of the device in the urethra was confirmed cystoscopically.

To aid in tracking cell populations 2′-deoxy-5-ethynyluridine (EdU) was given as an oral supplement (5 mg/kg) twice weekly. After 6-7 weeks of healing, terminal surgery was performed. After anesthesia was obtained, the urethra was identified, urethral pressures were collected, and animals were euthanized using intravenous pentobarbital (250 ml of 390 mg/ml).

The perineum was removed en-block and the urethra and bladder placed in formalin. Organs were weighed and representative samples collected. Blood draws were collected, and standard blood counts were performed (e.g., red blood cell count, white blood cell count, platelet count, albumin concentration) and chemistry obtained.

### Histological Procedure and Staining Quantification

Formalin fixed samples embedded in paraffin were processed on a microtome into 10 μm sections. Tissues were stained with hematoxylin and eosin stain (H&E) and Masson's Trichrome Stain and were assessed using a slide scanner (Axio Scan.Z1; Carl Zeiss AG, Oberkochen, Germany). Additional sections were de-paraffinized in sequential xylene washes and rehydrated in decreasing amounts of ethanol baths, finally being washed in water. Antigen retrieval was conducted by submerging sections into a sodium citrate buffer (10 mM sodium citrate, 0.05% Tween 20, pH 6.0) and boiling for 10 minutes. Sections were then permeabilized with blocking buffer (PBS+5% normal donkey serum, 5% BSA, 0.2% Triton-X) for an hour at room temperature. Primary antibodies against desmin (Abcam plc, Cambridge, United Kingdom), PD-L1 (Cell Signaling Technology, Inc., Danvers, MA), NF-κB p65 (Santa Cruz Biotechnology, Inc., Dallas, TX), M1 (LifeSpan BioSciences, Inc., Seattle, WA), and M2 macrophages (Abcam plc, Cambridge, United Kingdom) were diluted in blocking buffer and incubated with samples overnight at 4° C. (Table 1). Next, ALEXAFLUOR (Thermo Fisher Scientific, Inc., Waltham, MA) secondary antibodies were diluted 1:500 in blocking buffer and incubated with samples for one hour at room temperature. EdU was labeled according to the manufacturer's instructions using an imaging kit (CLICK-IT PLUS EdU AF647; Thermo Fisher Scientific, Inc., Waltham, MA). Following washes, mountant with DAPI (PROLONG GOLD antifade mountant; Thermo Fisher Scientific, Inc., Waltham, MA) was added to sections, a cover glass applied, and imaged on an inverted fluorescent microscope (AXIO OBSERVER, Carl Zeiss AG, Oberkochen, Germany) with variable fluorescence objectives. Gross images were taken with an iPhone (Apple, Inc., Cupertino, CA) camera.

Localization of the injury border zone of the injury was confirmed by Masson's Trichrome stain in order to correctly image the immunohistochemistry. Quantification of immunohistochemistry was assessed using ImageJ (Version 1.52a; Schneider et al., 2012, Nature Methods 9(7):671-675) macro scripts in a blinded manner. Tissues stained positive for desmin, PD-L1, and NF-κB p65 were quantified per percent tissue area of each respective image. Characterization of M2:M1 macrophage staining was conducted by quantifying MD and M2 area percentage per tissue area of each image. M1 quantification was defined by MD positive tissue minus M2 positive macrophage-stained tissue. Additionally, EdU positive count determined by object count per desmin-positive stained tissue area was quantified for determination of proliferative muscle.

### External Urethral Sphincter Pressure Collection

Urethral pressures were assessed using a portable anorectal manometry system (mCompass, Medspira, LLC, Minneapolis, Minnesota) with a five-channel modified pressure catheter using the squeeze mode of the biofeedback 1.01 software (FIG. 13D). Pressure recordings were performed in triplicate and were assessed at four time points: pre-injury (Day 0), post-injury (Day 0), prior to injection (Day 7), and prior to sacrifice (Day 42). Pressures readings were obtained along the length of the genitourinary tract starting in the vagina and extending to the bladder neck in 1 cm increments. At each point, pressures were collected for 10 seconds and three pressures along a given 10-second recording were averaged. Entry into the urethra was determined by a change in resistance with advancement of the pressure sensor and an associated rise in urethral pressures. Urethral entry and pressure measurement location was confirmed cystoscopically.

### Statistical Analysis

Data are reported as mean±standard error of the mean and interquartile range and statistical significance assessed with 2-tail student's T-test or assessed by single variable ANOVA with post-hoc Tukey Honestly Significant Difference Test comparing all experimental groups. Significance was set to alpha of 0.05.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method of treating injured skeletal muscle tissue, the method comprising applying a therapeutic composition to injured skeletal muscle tissue, the therapeutic composition comprising:
a purified exosome product (PEP); and
a pharmaceutically acceptable carrier.

2. The method of claim 1, wherein the PEP comprises spherical or spheroid exosomes having a diameter no greater than 300 nm.

3. The method of claim 1, wherein the PEP comprises spherical or spheroid exosomes having a mean diameter of 110 nm±90 nm.

4. The method of claim 3, wherein the PEP comprises spherical or spheroid exosomes having a mean diameter of 110 nm±50 nm.

5. The method of claim 4, wherein the PEP comprises spherical or spheroid exosomes having a mean diameter of 110 nm±30 nm.

6. The method of claim 1, wherein the PEP comprises:
from 1% to 20% CD63⁻ exosomes; and
from 80% to 99% CD63⁺ exosomes.

7. The method of claim 1, wherein the PEP comprises at least 50% CD63⁻ exosomes.

8. The method of claim 1, wherein the PEP comprises from 1×10¹¹ PEP exosomes to 1×10¹³ PEP exosomes.

9. The method of claim 8, wherein the PEP comprises from 1×10¹² PEP exosomes to 1×10¹³ PEP exosomes.

10. The method of claim 1 wherein the therapeutic composition further comprises a supportive matrix.

11. The method of claim 10, wherein the supportive matrix comprises a collagen scaffold.

12. The method of claim 1 wherein the therapeutic composition further comprises a tissue sealant or fibrin glue.

13. The method of claim 1 wherein the therapeutic composition forms a hydrogel after being applied to the injured skeletal muscle.

14. The method of claim 13, wherein the therapeutic composition forms a hydrogel when incubated at a temperature of 35° C. to 40° C.

15. The method of claim 13, wherein the therapeutic composition forms a hydrogel at least one minute after being applied to skeletal muscle.

16. The method of claim 1, wherein the therapeutic composition is applied in an amount effective to increase expression of NF-κB in the injured skeletal muscle tissue compared to injured skeletal muscle tissue treated without the therapeutic composition.

17. The method of claim 1 wherein the therapeutic composition is applied in an amount effective to increase expression of PD-L1 in the injured skeletal muscle tissue compared to injured skeletal muscle tissue treated without the therapeutic composition.

18. The method of claim 1 wherein the therapeutic composition is applied in an amount effective to increase polarization toward M2 macrophages in the injured skeletal muscle tissue compared to injured skeletal muscle tissue treated without the therapeutic composition.

19. The method of claim 1 wherein the injured skeletal muscle tissue comprises urethral sphincter muscle.

20. The method of claim 1 wherein at least a portion of the PEP exosomes comprise PD-L1, NF-κB, or both PD-L1 and NF-κB.

21. The method of claim 1, wherein at least a portion of the PEP exosomes comprise CD63, CD9, flotillin, or any combination of two or more of the foregoing.

22. The method of claim 1, wherein the therapeutic composition is applied in an amount effective to increase cellular proliferation in the injured skeletal muscle tissue compared to injured skeletal muscle tissue treated without the therapeutic composition.
